(19) United States
(12) Patent Application Publication
(10) Pub. No.: US 2017/0000375 A1
(43) Pub. Date: Jan. 5, 2017

(54) Magnetic Nanoparticle Detection and Separation by Magnetic Relaxation Time

(71) Applicant: Verily Life Sciences LLC, Mountain View, CA (US)

(72) Inventors: Vasiliki Demas, San Jose, CA (US); Vikram Singh Bajaj, Mountain View, CA (US); James Michael Higbie, Palo Alto, CA (US); Victor Marcel Acosta, Mountain View, CA (US); Michael Brundage, Mountain View, CA (US); Chinmay Belthangady, Mountain View, CA (US)

(21) Appl. No.: 14/788,882

(22) Filed: Jul. 1, 2015

(51) Int. Cl. A61B 5/05; A61B 5/145

(52) U.S. Cl. CPC A61B 5/05; A61B 5/145; A61B 2562/0223

(57) ABSTRACT

Wearable devices configured to detect the presence, concentration, number, or other properties of nanoparticles disposed in subsurface vasculature of a person are provided. The wearable devices are configured to magnetize the nanoparticles at an upstream location of subsurface vasculature and to detect, using a magnetometer, magnetic fields produced by the magnetized nanoparticles at a downstream location of subsurface vasculature. In some embodiments, the nanoparticles are configured to bind to an analyte of interest and detected properties of the magnetized nanoparticles can be used to determine the presence, concentration, or other properties of the analyte. Detecting magnetic fields produced by the magnetized nanoparticles can include detecting the fields directly, detecting an effect of the magnetic fields on nuclear magnetic spins of atoms proximate the magnetized nanoparticles, producing a time-varying magnetic field and detecting a time-varying magnetic field responsively produced by the magnetized nanoparticles, or some other method(s).

### BACKGROUND

Unless otherwise indicated herein, the materials described in this section are not prior art to the claims in this application and are not admitted to be prior art by inclusion in this section.

A number of scientific methods have been developed to detect, measure, and/or affect one or more analytes in a biological or other environment (e.g., a person's body). The one or more analytes could be any analytes that, when present in or absent from a person's body, or present at a particular concentration or range of concentrations, may be indicative of a medical condition or health state of the person. The one or more analytes could be substances whose distribution, action, or other properties, interactions, or activities throughout an animal's body is of scientific or medical interest. The one or more analytes could include pharmaceuticals or other substances introduced into the biological or other environment to effect some chemical or biological process. The one or more analytes could be present in living or nonliving human or animal tissue, and could be detected, measured, or affected in an in vivo, ex vivo, in vitro, or some other type of sample. The one or more analytes could include enzymes, reagents, hormones, proteins, drugs, nanoparticles, pharmaceuticals, cells or other molecules.

### SUMMARY

Some embodiments of the present disclosure provide a device including: (i) a magnetometer that is configured to be positioned proximate to a first location of subsurface vasculature and that is configured to detect magnetic fields at the first location; (ii) a magnetic flux source that is configured to be positioned proximate to a second location of the subsurface vasculature and that is configured to magnetize nanoparticles in the in the subsurface vasculature that are proximate the second location, wherein the second location is located upstream from the first location relative to a direction of blood flow in the subsurface vasculature; and (iii) a controller that is operably coupled to the magnetometer and that includes a computing device programmed to perform controller operations. The controller operations include: (a) operating the magnetometer to detect a magnetic field at the first location; and (b) determining a property of magnetized nanoparticles based on the detected magnetic field, wherein the magnetized nanoparticles include nanoparticles that were magnetized by the magnetic flux source at the second location and that traveled to the first location.

Some embodiments of the present disclosure provide a device including: (i) means for detecting magnetic fields at a first location of subsurface vasculature; (ii) means for magnetizing nanoparticles that are proximate a second location of the subsurface vasculature, wherein the second location is located upstream from the first location relative to a direction of blood flow in the subsurface vasculature; and (iii) controller means that are operably coupled to the means for detecting magnetic fields. The controller means are configured to perform controller operations including: (a) operating the means for detecting magnetic fields to detect a magnetic field at the first location; and (b) determining a property of magnetized nanoparticles based on the detected magnetic field, wherein the magnetized nanoparticles include nanoparticles that were magnetized by the magnetic flux source at the second location and that traveled to the first location.

Some embodiments of the present disclosure provide a method including: (i) magnetizing, using a magnetic flux source, nanoparticles in a first location of subsurface vasculature; (ii) detecting, using a magnetometer, a magnetic field at a second location of the subsurface vasculature, wherein the second location is located downstream from the first location relative to a direction of blood flow in the subsurface vasculature; and (iii) determining a property of magnetized nanoparticles based on the detected magnetic field, wherein the magnetized nanoparticles include nanoparticles that were magnetized by the magnetic flux source at the first location and that traveled to the second location.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1A is a side cross-sectional view of nanoparticles in a portion of subsurface vasculature and a device positioned proximate to the portion of subsurface vasculature, in accordance with an example embodiment.

FIG. 1B illustrates an example output over time of a magnetic sensor of the device of FIG. 1A as magnetized nanoparticles in the portion of subsurface vasculature of FIG. 1A move through the portion of subsurface vasculature.

FIG. 2 is a side cross-sectional view of nanoparticles in a portion of subsurface vasculature and a device positioned proximate to the portion of subsurface vasculature, in accordance with an example embodiment.

FIG. 3 is a side cross-sectional view of nanoparticles in a portion of subsurface vasculature and a device positioned proximate to the portion of subsurface vasculature, in accordance with an example embodiment.

FIG. 4 is a side cross-sectional view of nanoparticles in a portion of subsurface vasculature and a device positioned proximate to the portion of subsurface vasculature, in accordance with an example embodiment.

FIG. 5A is a side cross-sectional view of nanoparticles in a portion of subsurface vasculature and a device positioned proximate to the portion of subsurface vasculature during a first period of time, in accordance with an example embodiment.

FIG. 5B is a side cross-sectional view of the nanoparticles in the portion of subsurface vasculature of FIG. 5A and the device positioned proximate to the portion of subsurface vasculature of FIG. 5A during a second period of time, in accordance with an example embodiment.

FIG. 6 illustrates an example frequency spectrum of an output of a magnetic sensor.

FIG. 7 is a is a side cross-sectional view of nanoparticles in a system configured to separate the nanoparticles according to a magnetic property of the nanoparticles

FIG. 8 is perspective view of an example device.

FIG. 9 is an illustration of a number of wearable devices in communication with a server.

FIG. 10 is a block diagram of an example device.

FIG. 11 is a flowchart of an example method.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

### I. OVERVIEW

Nanoparticles can be configured to be magnetizable (e.g., to develop a magnetic moment in response to an external magnetic field and to retain such a magnetic moment for some specified period of time) and to selectively bind with an analyte of interest. Nanoparticles configured in this way can enable manipulation of, detection of, or other interactions with the analytes by applying magnetic forces to the magnetized nanoparticles. Additionally or alternatively, an analyte of interest could be intrinsically magnetic and/or magnetizable, or could be an engineered analyte (e.g., a pharmaceutical) that includes a magnetic property and/or that is bound to a magnetizable nanoparticle and that can be introduced into an environment according to an application. Detecting the magnetic field produced by such magnetized nanoparticles could allow for the determination of the amount (e.g., concentration, number), distribution, or other properties of the analyte of interest in the biological environment. For example, the magnetic field produced by such analyte-binding magnetized nanoparticles in a portion of subsurface vasculature could be detected (e.g., using one or more magnetometers disposed in a wearable device mounted proximate to the portion of subsurface vasculature) and used to determine the number and/or concentration of the analyte in the blood in the portion of subsurface vasculature.

In some examples, permanently magnetized nanoparticles in vasculature of a body (e.g., nanoparticles that include ferromagnetic material or other material capable of being permanently magnetized) could, as a result of mutual magnetic attraction, aggregate into clumps. Such clumps could have a sufficient size or other properties such that the clumps of magnetized nanoparticles block small-diameter blood vessels. Further, in examples wherein such permanently magnetized nanoparticles are configured to selectively interact with (e.g., to reversibly bind to) an analyte, clumping of the nanoparticles could prevent interaction between the nanoparticles and the analyte.

In some examples, nanoparticles in vasculature of a body could be magnetizable and further could be configured such that magnetization of the nanoparticles (e.g., by application of an external magnetic field) is temporary, i.e., that the magnetization of such a nanoparticle could decay, reverse, or otherwise diminish or change over time. For example, such nanoparticles could include paramagnetic materials, superparamagnetic materials, or other materials or structures such that a state of magnetization of the nanoparticles decreases over time. Such nanoparticles could be configured to have a specified magnetic relaxation time (e.g., a magnetic relaxation time within a specified range of relaxation times, e.g., between approximately 1 second and approximately 2 seconds) or to have specified some other measure of the time-dependence of the decrease of the magnetization of such a nanoparticle over time subsequent to being magnetized.

In such examples, the nanoparticles could be magnetized and a property (e.g., a direction, magnitude, or other property of a magnetic field produced by) of the magnetized nanoparticles could be detected. For example, the nanoparticles could be magnetized (e.g., by a permanent magnet or other magnetic flux source) in an upstream location of vasculature (upstream relative to a flow of blood in the vasculature) and the magnetized nanoparticles could be detected (e.g., by a magnetometer) in a second, downstream location of vasculature after the magnetized nanoparticles have flowed, with the blood in the vasculature, from the upstream location to the downstream location. The magnetization of the magnetized nanoparticles could subsequently decrease such that the nanoparticles substantially do not clump.

Nanoparticles having a magnetic relaxation time (or some other measure of the decrease of the magnetization of magnetized nanoparticles over time) within a specified range of relaxation times could be produced in a variety of ways. In some examples, a plurality of individual nanoparticles having a range of relaxation times could be sorted or partitioned such that individual nanoparticles having relaxation times within the specified range are separated from the remainder of the plurality of individual nanoparticles. In some examples, this could include disposing the plurality of individual nanoparticles in a flowing carrier fluid, magnetizing the plurality of individual nanoparticles (e.g., using a permanent magnet) in an upstream location, applying a separating magnetic force to the magnetized nanoparticles (e.g., using a permanent magnet) in a downstream location, and collecting and/or partitioning the separated nanoparticles (e.g., using a forked tube or other means for separating a fluid flow). As a result, individual nanoparticles of the plurality of individual nanoparticles that have a relaxation time greater than a specified value (e.g., a specified value related to a distance between the upstream location and the downstream location and a rate of flow of the carrier fluid) could be separated from nanoparticles of the plurality of individual nanoparticles that have a relaxation time less than the specified value. Such a separation could be performed a number of times, e.g., to first separate nanoparticles having relaxation times less than an upper end of a specified range of relaxation times and to second separate nanoparticles having relaxation times greater than a lower end of the specified range of relaxation times. Additionally or alternatively, a relaxation time of the nanoparticles could be related to a size (e.g., diameter) of the nanoparticles, and the nanoparticles could be separated according to size to provide separation of individual nanoparticles having specified relaxation times.

Such nanoparticles having specified magnetic relaxation times or otherwise configured to be temporarily magnetizable could be used as described herein to be detected subsequent to being magnetized while generally being non-magnetized, e.g., to prevent clumping of the nanoparticles. Further, detection of such nanoparticles could be provided by the nanoparticles having such specified magnetic relaxation times. For example, a change in magnetization of such nanoparticles over time could be detected (e.g., using two magnetometers configured to detect magnetic fields in respective different locations of vasculature downstream from a magnetic flux source configured to magnetize the nanoparticles) and used to provide an improved measure of the number or amount of the magnetized nanoparticles in the vasculature. This could include, for example, comparing a magnetic field detected at a first location at which the magnetized nanoparticles remain magnetized (e.g., due to proximity to an upstream magnetic flux source) to a magnetic field detected at a second location at which the magnetized nanoparticles have become de-magnetized (e.g., due to a greater distance from the upstream magnetic flux source). In some examples, first and second pluralities of nanoparticles having magnetic relaxation times within respective first and second non-overlapping ranges of relaxation times could be detected in such a way (i.e., by detecting magnetic fields in multiple different locations of vasculature downstream from a nanoparticle-magnetizing magnetic flux source). For examples, this could provide for the detection of amounts of first and second analytes with which the first and second pluralities of nanoparticles are, respectively, configured to selectively interact (e.g., bind)

Magnetizable and/or magnetic nanoparticles as described herein may be made of and/or wholly or partially coated by an inert material, such as polystyrene, and can have a diameter that is less than about 20 micrometers. In some embodiments, the particles have a diameter on the order of about 5 nm to 1 μm. In further embodiments, one or more particles of magnetic and/or magnetizable material of a nanoparticle (e.g., particles of superparamagnetic iron oxide) may be embedded in a substrate of non-magnetic material (e.g., polystyrene). In some examples, the size and/or a distribution of sizes of such nanoparticles and/or particles of magnetizable material thereof could be specified to control a magnetic or other property of the nanoparticles, e.g., to control a magnetic relaxation time, coercivity, remanence, susceptibility, type of magnetic behavior (e.g., superparamagnetism, ferromagnetism, ferrimagnetism, paramagnetism), hysteresis, or other property of the nanoparticles. For example, a particle of magnetizable material of a nanoparticle could have a size between approximately 10 nanometers and approximately 20 nanometers e.g., such that the particle of magnetizable material comprises a single magnetic domain. In another example, a particle of magnetizable material could have a size of approximately 20 nanometers such that the particle of magnetizable material has a magnetic relaxation time of approximately 1 second. The nanoparticles and/or particles of magnetic and/or magnetizable material thereof may be formed from a paramagnetic, ferrimagnetic, ferro-magnetic, or super-paramagnetic material or any other material that responds to a magnetic field and that exhibits a magnetization that decreases over time (e.g., at a specified relaxation rate).

Those of skill in the art will understand a “particle” in its broadest sense and that it may take the form of any fabricated material, a molecule, cryptophane, a virus, a phage, etc. Further, a nanoparticle may be of any shape, for example, spheres, rods, non-symmetrical shapes, etc. Further, the nanoparticles can be configured to selectively bind to one or more analytes (e.g., chemicals, hormones, peptides, DNA or RNA fragments, cells). Such particles could be introduced into an environment that contains the one or more analytes (e.g., into the blood of a body, into a portion of subsurface vasculature of a body, into a fluid of a natural environment, water treatment process, pharmaceutical process, or some other environment of interest). Alternatively, the one or more analytes and/or a fluid or other material containing the one or more analytes could be extracted (e.g., from an environment of interest) and introduced into another environment into which the nanoparticles have been or could be introduced.

Detection of magnetic fields produced by magnetized nanoparticles could provide a variety of applications. The magnetized nanoparticles could be configured to selectively interact with (e.g., to bind to) one or more analytes of interest. Detection of the magnetic fields produced by the magnetized nanoparticles could allow for the determination of one or more properties of the analytes of interest, e.g., an amount of the analytes, a concentration of the analytes, a number of the analytes (e.g., a number of cancer cells in a portion of subsurface vasculature and/or in the blood circulation of a body), a property of the analytes, or some other information about the analytes. Detection of magnetic fields produced by magnetized nanoparticles could allow the determination of the orientation and/or location of the magnetized nanoparticles (e.g., by detecting a magnitude and/or direction of the produced magnetic field at one or more locations proximate to (e.g., outside of) the environment of interest, e.g., outside skin proximate a portion of subsurface vasculature), a degree of aggregation of the magnetized nanoparticles (e.g., by detecting a magnitude of the produced magnetic field, by detecting a property of change over time of the produced magnetic field), or the detection of some other property of the magnetized nanoparticles.

Such determined properties of the magnetized nanoparticles could be related to properties of the analytes of interest. For example, multiple magnetized nanoparticles could bind to a single instance of an analyte (e.g., to a single cancer cell) such that detection of an aggregate of magnetized nanoparticles (e.g., detection of a large amplitude magnetic field produced by such aggregated magnetized nanoparticles) allows for the determination that the single instance of the analyte is present (e.g., that a cancer cell is present in a portion of subsurface vasculature). Other properties of a detected magnetic field produced by magnetized nanoparticles could be used in similar or different ways to determine properties of one or more analytes in an environment of interest.

One or more properties of the analyte could be related to a medical condition of a human or animal containing the analyte. In some examples, the analyte could have a medical or other effect on the human or animal (e.g., the analyte is a toxin, the analyte is a pharmaceutical, the analyte is a cancer cell), and detecting magnetic fields produced by magnetized nanoparticles bound to the analyte could allow detection or determination of a medical condition of the human or animal. For example, the analyte could be a cancer cell, and detection of the magnetic fields produced by magnetized nanoparticles in the blood could allow the detection of an amount of the cancer cells in the blood, a stage of the cancer, that the cancer has entered or left remission, or some other information or health state. Other applications and environments containing such nanoparticles are anticipated.

Magnetized nanoparticles could be detected in a variety of ways. A direction, magnitude, property of change over time, or some other property of the produced magnetic fields could be detected. Such detection could include operating one or more magnetometers (i.e., devices or components configured to detect one or more properties of a magnetic field, e.g., magnitude, direction, magnitude in a specified direction) to directly detect produced magnetic fields at one or more respective locations proximate to (e.g., outside of) an environment of interest that contains the magnetized nanoparticles. For example, a body-mountable device including one or more magnetometers could be mounted to a skin surface proximate a portion of subsurface vasculature such that the one or more magnetometers can detect magnetic fields produced by the magnetized nanoparticles in the portion of subsurface vasculature. In some examples, the detected produced magnetic field could be produced in response to an oscillating or otherwise time-varying field produced in the environment of interest. For example, an oscillating magnetic field could be produced, and an oscillating magnetic field responsively produced by the magnetized nanoparticles in the environment (e.g., an oscillating magnetic field at a harmonic of the produced oscillating magnetic field) could be detected.

In some examples, magnetic field produced by magnetized nanoparticles could be detected indirectly, e.g., by magnetically or otherwise detecting a property that is related to and/or affected by the magnetic field produced by the magnetized nanoparticles. For example, a precession frequency or other information about magnetic spins of atomic nuclei in the environment (e.g., a T2* spin relaxation time of packets of atomic nuclei having polarized magnetic spins) that is related to the magnetized nanoparticles (e.g., that is changed by inhomogeneities in the Earth's magnetic field that are produced by the magnetized nanoparticles) could be detected. This could include polarizing the magnetic spins of hydrogen atoms or other nuclei in a first location of vasculature (e.g., using a permanent magnet or other magnetic flux source located at a second location of vasculature that is upstream from the first location of vasculature), rotating the magnetic spins of atomic nuclei in the first location of vasculature by producing a time-varying magnetic field in the first location (e.g., using coils of a pulse emitter), and detecting time-varying magnetic fields produced by atomic nuclei in the first location in response to the rotation of the magnetic spins of the atomic nuclei (e.g., using a magnetometer).

Magnetometers used to detect magnetic fields as described herein could be configured to detect magnetic fields that have very small magnitudes. For example, a magnetometer used to detect magnetic fields produced by magnetized nanoparticles and/or produced by precessing atomic spins could be configured to have a sensitivity such that the magnetometer can detect changes in a measured magnetic field (e.g., a magnetic field at a location less than approximately 1 centimeter outside a portion of subsurface vasculature) of less than approximately 10 femtoteslas. Magnetometers could include superconducting quantum interference devices (SQUIDs), spin-exchange relaxation-free (SERF) magnetometers, multi-pass optical atomic magnetometers, inductive loops or coils or other antenna structures, spin precession magnetometers, or some other magnetic-field-detecting components or devices. Further, the magnetic fields (e.g., magnetic fields produced by magnetized nanoparticles) could be detected at more than one location (e.g., by more than one magnetometer) to allow for detection of properties of the magnetized nanoparticles (e.g., to detect a speed of movement of the nanoparticles in a portion of subsurface vasculature) and/or to allow a background magnetic field (e.g., a magnetic field present in the environment of interest that is not produced by and/or related to the magnetized nanoparticles, e.g., that is produced by the Earth, that is produced by electronic devices, that is produced by other magnetic and/or magnetized materials in or proximate to the environment of interest).

The effects of a background magnetic field (e.g., a magnetic field produced by electronics or magnetic materials proximate to and/or within an environment of interest, a magnetic field produced by the Earth) could be mitigated or compensated for in a variety of ways. In some examples, a system could include two or more magnetometers configured to detect magnetic fields at two or more respective locations. In such examples, a magnetic field produced by magnetic particles in the environment of interest could be determined by determining a difference between the magnetic fields detected by two of the two or more magnetometers. In some examples, a system could include magnetic shims, magnetic shielding materials, permanent magnets, electromagnets, or other means for changing and/or controlling a magnetic field detected by a magnetometer. Such means could be used to reduce a background magnetic field detected at a location by the magnetometer (e.g., to cancel a magnetic field produced by the Earth and detected by the magnetometer) and/or to cancel and/or reduce an inhomogeneity of a magnetic field produced by a component of the system or by some other system (e.g., a magnetic field produced by an electromagnet that is configured to magnetize nanoparticles). Such means could be operated based on a magnetic field detected by a magnetometer (e.g., to zero the output of the magnetometer), based on a magnetic field detected by another magnetometer (e.g., to reduce the magnetic field present at the location of a SERF magnetometer based on a magnetic field detected by a Hall effect magnetometer located proximate to the SERF magnetometer), or based on some other information or consideration.

Magnetometers and magnetic flux sources configured as described herein could be included as part of a variety of systems or devices and configured to magnetize nanoparticles and to detect magnetic fields produced by such magnetized nanoparticles present in a variety of flow environments according to a variety of applications. In some examples, one or more magnetometers, magnetic flux sources, or other components could be included in a body-mountable device configured to be mounted to a skin surface and to magnetize nanoparticles and to detect magnetic fields produced by such magnetized nanoparticles in a portion of subsurface vasculature proximate the skin surface. Additionally or alternatively, magnetometers and/or magnetic flux sources as described herein could be included in handheld, desktop, wall- or floor-mounted devices, or some other type of device or system. Such systems could be configured to magnetize nanoparticles and to detect magnetic fields produced by such magnetized nanoparticles disposed in natural environments (e.g., portions of subsurface vasculature, fluids of a lake, stream, or other natural outdoor environment), ex vivo and/or in vitro environments (e.g., fluids contained in a sample container), artificial environments (e.g., a fluid or other volume of a pharmaceutical or industrial process), or some other environment of interest. Nanoparticles could be introduced into the environment of interest (e.g., injected into a portion of subsurface vasculature), naturally present in the environment of interest, introduced into a sample extracted from an environment of interest, or otherwise disposed relative to an environment of interest.

Further, note that methods described herein to detect properties (e.g., presence, location, orientation, number, degree of aggregation, state of binding to an analyte) of magnetized nanoparticles could be applied to detect such properties of nanoparticles that are permanently magnetic, e.g., that maintain a magnetic dipole moment without being recently exposed to a magnetic flux source. Such permanently magnetized nanoparticles could include magnetic materials that are permanently magnetizable, could include particles of a ferromagnetic, ferromagnetic, or otherwise magnetic material that includes multiple magnetic domains or that is otherwise configured to be permanently magnetized or to otherwise maintain a magnetic moment for a protracted period of time.

It should be understood that the above embodiments, and other embodiments described herein, are provided for explanatory purposes, and are not intended to be limiting.

Further, the term “medical condition” as used herein should be understood broadly to include any disease, illness, disorder, injury, condition or impairment—e.g., physiologic, psychological, cardiac, vascular, orthopedic, visual, speech, or hearing—or any situation requiring medical attention.

### II. ILLUSTRATIVE MAGNETIZED PARTICLES AND MAGNETIZATION AND DETECTION THEREOF

Magnetic fields produced by magnetized nanoparticles in an environment of interest can be detected (e.g., by one or more magnetometers located within and/or proximate to the environment of interest) and used to determine the location, amount (e.g., number, concentration), orientation, velocity, degree of aggregation, or other properties of the magnetized nanoparticles in the environment of interest and/or to determine properties of the environment of interest. Such magnetized nanoparticles could be magnetized in the environment of interest (e.g., a magnetic flux source could generate a magnetic flux sufficient to magnetize the nanoparticles). Alternatively, the nanoparticles could be magnetized in a location that is different from the region of the environment of interest where the magnetic field is detected (e.g., the nanoparticles could be magnetized in a first portion of subsurface vasculature that is upstream from a second portion of subsurface vasculature proximate to which a magnetometer is disposed to detect magnetic fields produced by magnetized nanoparticles that have flowed from the first location to the second location).

The environment of interest could include artificial environments (e.g., a fluid of an industrial process, a fluid of a chemical or pharmaceutical process) or natural environments (e.g., a lake, a river, a marsh, blood in vasculature of an animal). For example, the nanoparticles could be disposed in blood in a portion of subsurface vasculature of a human. The nanoparticles could be permanently magnetic (e.g., could include particles of ferromagnetic material having multiple magnetic domains) or could be magnetizable when exposed to a magnetic field (e.g., could be paramagnetic or superparamagnetic) or to some other factor. In some examples, the nanoparticles can be configured to bind to an analyte of interest and magnetic fields produced by the magnetized nanoparticles could be detected to determine the location, amount (e.g., number, concentration), state of binding to one or more magnetized nanoparticles, or other properties of the analyte of interest.

The magnetic field produced by one or more magnetized nanoparticles can be detected at one or more locations in space. The direction, magnitude, and/or other properties of the produced magnetic field at a particular location can be related to the location and/or orientation of the one or more magnetized nanoparticles relative to the particular location, the magnitude of the permanent and/or induced magnetic dipole moment of the magnetized nanoparticles, magnetic properties of materials proximate the particular location, or other factors. A magnetic field at the particular location (e.g., a direction and/or magnitude of a magnetic field detected by, e.g., a magnetometer) could be related to the magnetic field of the earth, magnetic fields produced by electronics or other devices proximate the particular location, magnetic and/or electromagnetic fields produced by atomic magnetic spins that are precessing in a magnetic field (e.g., a magnetic field produced by the Earth and/or by one or more magnetized nanoparticles), magnetized or otherwise magnetic materials proximate the particular location, or other factors in addition to the magnetic field produced by the one or more magnetized nanoparticles.

The magnetized nanoparticles could produce a magnetic field intrinsically. For example, each nanoparticle could include magnetized ferromagnetic, ferrimagnetic, paramagnetic, superparamagnetic, or otherwise magnetized materials and/or each nanoparticle could include superparamagnetic materials that become spontaneously magnetized. In such examples, this produced magnetic field could be detected at one or more locations (e.g., by a magnetometer) and used to determine one or more properties of the nanoparticles. For example, detecting a magnetic field (e.g., detecting a magnitude, direction, change over time, or other properties of the magnetic field) at a particular location could provide information about the location, orientation, number, state of binding to an analyte, degree of magnetization or other magnetic state, degree of aggregation (e.g., aggregation proximate to an instance of an analyte to which the nanoparticles are configured to bind), or some other information about magnetized nanoparticles proximate the particular location. Additionally or alternatively, the magnetic field produced by the nanoparticles could be induced by an external static and/or time-varying magnetic field or other applied energy or field. The nanoparticles could include a coating and/or be composed of a material that is biocompatible and/or specified to interact in some way with biological and/or chemical elements in an environment of interest (e.g., to interact specifically with an analyte of interest).

The nanoparticles may each include magnetic materials having a coercivity, remanence, susceptibility, permanent magnetic moment, or other magnetic property such that the nanoparticles can produce a magnetic field (e.g., by being magnetized, by reflecting or otherwise interacting with a time-varying electromagnetic field) that could be detected by a magnetometer proximate to the nanoparticles. In some examples, this could include the nanoparticles each including a single piece of magnetic material, e.g., a single particle or crystal of a ferromagnetic, ferrimagnetic, paramagnetic, superparamagnetic, or otherwise magnetic material. Such a magnetic material of a nanoparticle could be coated by an inert material, such as polystyrene. The nanoparticles could be similar (e.g., could each be similarly sized) or could vary, e.g., the size of the nanoparticles or some other properties of the nanoparticles could vary according to a distribution. For example, the nanoparticles could be configured to be magnetized by an external magnetic field and to have a degree of magnetization that decreases and/or reverses over time. For example, the nanoparticles could have magnetic relaxation times that are within a specified range of relaxation times (e.g., between approximately 100 milliseconds and approximately 1 second, or between approximately 1 second and approximately 2 seconds).

The nanoparticles could have an overall size and/or shape specified according to an application. For example, the nanoparticles could have a size and/or shape such that the nanoparticles can be transported in blood in the vasculature of a body without causing blockages and/or such that the nanoparticles, when magnetized (e.g., by application of a magnetic field by a magnetic flux source), produce a magnetic field having a sufficiently high magnitude to be detected by one or more magnetometers proximate the magnetized nanoparticles (e.g., to be detect by a magnetometer located outside of a portion of subsurface vasculature containing the magnetized nanoparticles, e.g., from approximately a millimeter to approximately a centimeter away from the magnetized nanoparticles). In some examples, the nanoparticles can have a diameter that is less than about 20 micrometers. In some embodiments, the nanoparticles have a diameter on the order of about 5 nm to 1 μm.

In some examples, the nanoparticles could have a magnetic relaxation time that is less than some specified value such that the magnetization decays, changes orientation, changes sign, or otherwise decreases in an amount of time related to the magnetic relaxation time. For example, the nanoparticles could have magnetic relaxation times between approximately 1 second and approximately 2 seconds. In another example, the nanoparticles could have magnetic relaxation times between approximately 100 milliseconds and approximately 1 second. In some examples, the magnetic relaxation time could be specified to be sufficiently long that the nanoparticles could substantially remain magnetized during a time required to flow from a first region at which the nanoparticles are magnetized (e.g., by a magnetic flux source) to a second region at which a magnetometer detects, directly or indirectly, a magnetic field produced by the still-magnetized nanoparticles. Such a specified magnetic relaxation time could be specified based on a distance between a magnetometer and a magnetic flux source configured to magnetize the nanoparticles, a flow rate or velocity of the nanoparticles (e.g., a flow rate of blood in a portion of subsurface vasculature, e.g., approximately 1 centimeter per second).

In some examples, the size of the nanoparticles could be related to the magnetic relaxation time, such that specifying a range of magnetic relaxation times of the nanoparticles could include specifying a size of the nanoparticles and/or of elements of magnetic material (e.g., a particle of superparamagnetic iron oxide) thereof. For example, a range of sizes of the nanoparticles (and/or of an element of magnetic material thereof) between approximately 10 nanometers and approximately 20 nanometers could correspond to a range of magnetic relaxation times between approximately 1 nanosecond and approximately 1 second.

In further embodiments, nanoparticles of magnetic material (e.g., particles of ferromagnetic, ferromagnetic, paramagnetic and/or superparamagnetic material) and/other small particles on the order of 10-100 nanometers in diameter may be assembled to form larger “clusters” or “assemblies” on the order of 1-10 micrometers. Further, the a magnetic relaxation time(s), arrangement, relative location and/or orientation, number, or other properties of such particles of magnetic material comprising a nanoparticle could be specified such that the nanoparticle is magnetizable and has a magnetic relaxation time within some specified range of relaxation times.

Those of skill in the art will understand a “particle” in its broadest sense and that it may take the form of any fabricated material, a molecule, cryptophan, a virus, a phage, etc. Further, a nanoparticle may be of any shape, for example, spheres, rods, non-symmetrical shapes, etc. In some examples, a magnetic material of the nanoparticles can include a paramagnetic, super-paramagnetic or ferromagnetic material or any other material that responds to a magnetic field. In some examples, the nanoparticles can include a magnetic moiety (e.g., an organic molecule that has a magnetic and/or magnetizable molecular orbital). Further, the particles can be configured to selectively bind to one or more analytes (e.g., chemicals, hormones, peptides, DNA or RNA fragments, cells). In some examples, the nanoparticles could be considered to include other elements (e.g., analytes, other magnetic or non-magnetic particles) bound to the nanoparticles. Other embodiments of nanoparticles are anticipated.

In some examples, the nanoparticles are functionalized to selectively interact with an analyte of interest. The nanoparticles can be functionalized by covalently attaching a bioreceptor designed to selectively bind or otherwise recognize a particular analyte (e.g., a clinically-relevant analyte, e.g., a cancer cell). For example, nanoparticles may be functionalized with a variety of bioreceptors, including antibodies, nucleic acids (DNA, siRNA), low molecular weight ligands (folic acid, thiamine, dimercaptosuccinic acid), peptides (RGD, LHRD, antigenic peptides, internalization peptides), proteins (BSA, transferrin, antibodies, lectins, cytokines, fibrinogen, thrombin), polysaccharides (hyaluronic acid, chitosan, dextran, oligosaccharides, heparin), polyunsaturated fatty acids (palmitic acid, phospholipids), or plasmids. The functionalized nanoparticles can be introduced into a portion of subsurface vasculature of a person or other environment of interest by injection, ingestion, inhalation, transdermal application, or in some other manner.

A clinically-relevant analyte could be any substance that, when present in the blood of a person or animal, or present at a particular concentration or range of concentrations and/or in a certain amount, may be indicative and/or causative of an adverse medical condition. For example, the clinically-relevant analyte could be an enzyme, hormone, protein, other molecule, or even whole or partial cells. In one relevant example, certain proteins have been implicated as a partial cause of Parkinson's disease. Thus, the development of Parkinson's disease might be prevented or retarded by providing nanoparticles functionalized with a bioreceptor that will selectively bind to this target. The nanoparticles could be magnetic and/or could be magnetized, and a magnetic field produced by the magnetized nanoparticles may then be detected, using one or more systems or devices as described herein (e.g., a magnetometer in a wearable device mounted to an external body surface proximate to a portion of subsurface vasculature), to detect a property (e.g., a concentration, a presence) of the bound protein (e.g., to inform a treatment, to adjust a dosage of a drug). As a further example, the analyte could be a cancer cell. By detecting a magnetic field produced by magnetized nanoparticles configured to selectively interact with the cancer cells, the progress of cancer (e.g., remission, stage) may be quantified and used to inform some treatment or other action (e.g., to begin chemotherapy, to set a dosage of a chemotherapy drug).

In some examples, nanoparticles configured to selectively interact with (e.g., bind to) an analyte of interest could be used to provide some additional applications. For example, an attractive magnetic force could be applied to magnetized nanoparticles to collect, extract, or otherwise manipulate the analyte. Additionally or alternatively, the nanoparticles could be used to modify or destroy the analyte of interest, e.g., by transducing an electromagnetic energy directed toward the nanoparticles (e.g., RF energy) into heat to denature or otherwise modify or destroy the analyte. In some examples, such operations (e.g., emission of an optical, RF, thermal, acoustical, or other type of energy to modify or destroy an analyte of interest) could be performed in response to determining some information about the analyte (e.g., determining that an instance of the analyte is proximate to a magnetometer of a device, and further within an area of effect of an energy emitter of the device) based on a detected magnetic field produced by the magnetized nanoparticles.

Magnetic fields produced by magnetized nanoparticles and detected at one or more locations (e.g., by magnetometers disposed at the one or more locations) can be used in a variety of ways to detect properties of the magnetized nanoparticles and/or to detect properties of an analyte of interest with which the magnetized nanoparticles are configured to selectively interact. For example, a direction, velocity, orientation, angular velocity, magnetic moment, degree of magnetization, degree of aggregation, or other properties of one or more magnetized nanoparticles could be determined based on a magnetic field detected at one or more locations. Further, the presence, concentration, location, velocity, or other properties of the analyte could be determined based on the detected magnetic field and/or based on the determined properties of the magnetized nanoparticles. For example, the nanoparticles could be configured such that a plurality of nanoparticles could selectively interact with (e.g., bind to) a single instance of the analyte of interest. In such examples, the detection and/or determination that a plurality of magnetized nanoparticles are aggregated (e.g., proximate each other) could be used to determine that an instance of the analyte is located proximate the aggregated magnetized nanoparticles. Other properties of a detected magnetic field and/or determined properties of the magnetized nanoparticles could be used to determine properties (e.g., location, number, concentration) of the analyte. For example, a velocity, angular velocity, magnetic property, or other property of the magnetized nanoparticles could be related to interaction between the magnetized nanoparticles and the analyte.

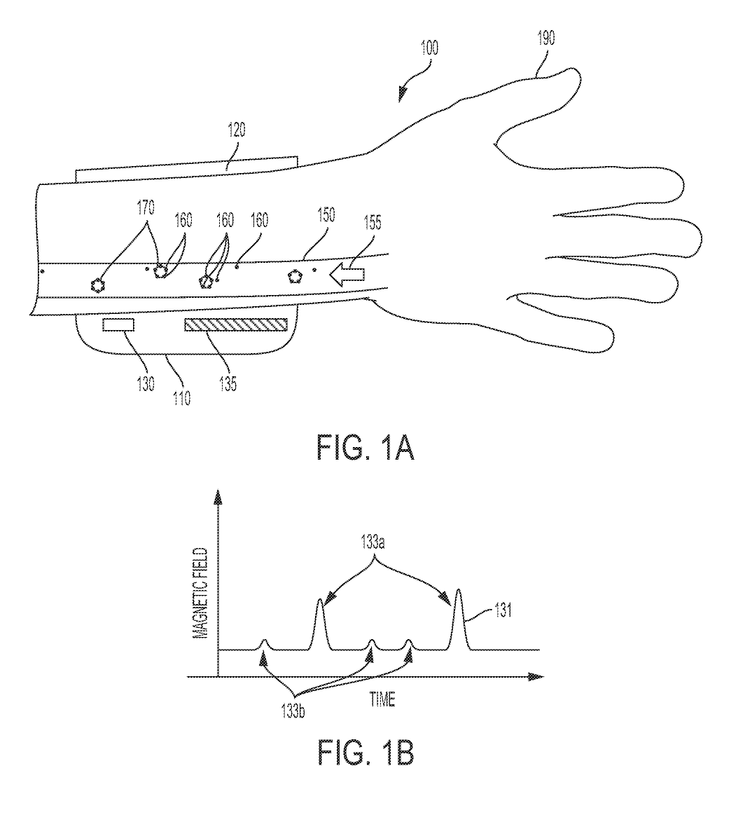

FIG. 1A illustrates example nanoparticles 160 and an analyte of interest 170 with which the nanoparticles 160 are configured to selectively interact disposed in a blood vessel 150 (i.e., a portion of subsurface vasculature). In this example, disposed in blood vessel 150 are instances of an analyte 170 (e.g., a cell), in which each instance of analyte 170 is bound to several nanoparticles 160. Also disposed in blood vessel 150 are unbound nanoparticles 160. The blood vessel 150 is located in an arm 190 and contains blood that is flowing (direction of flow indicated by the arrow 155). A body-mountable device 100 includes a housing 110 mounted outside of or otherwise proximate to the blood vessel 150 by a mount 120 configured to encircle the arm 190. The body-mountable device 100 include a magnetic flux source 135 (e.g., a permanent magnet, an electromagnet) configured to magnetize the nanoparticles 160 in the blood vessel 150 that are proximate the magnetic flux source 135 (e.g., that are within a first location of subsurface vasculature that is proximate the magnetic flux source 135).

The body-mountable device 100 further includes a magnetometer 130 disposed in the housing 110 and configured to detect a magnetic field at a location outside of the arm 190 (e.g., at a location within the magnetometer 130). The location of detected magnetic field is downstream, relative the flow of blood 155, from the location at which the nanoparticles 160 are magnetized by the magnetic flux source 135 (e.g., a second location of subsurface vasculature that is proximate the magnetometer 130 and that is downstream from the first location of subsurface vasculature). The magnetic field detected by the magnetometer 130 could include magnetic fields produced by the nanoparticles 160 that are magnetized and that are proximate the magnetometer 130, a magnetic field produced by the Earth, a magnetic field produced by electronics and/or electrical wiring (e.g., a magnetic field produced by an electromagnet, by other electronics of the body-mountable device 100, a magnetic field produced by a nearby automobile), an electromagnetic and/or magnetic field produced by precessing atomic magnetic moments (e.g., a time-varying magnetic field produced by precessing magnetic spins of hydrogen nuclei in water or other molecules in the blood vessel 150), a magnetic field produced and/or affected by a magnet or other magnetic material, and or some other magnetic fields and/or combinations of magnetic fields.

A distance between the magnetic flux source 135 and the magnetometer 130 could be specified based on a variety of factors according to an application. In some examples, a distance between the magnetic flux source 135 and the magnetometer 130 could be specified to be greater than a specified distance such that a degree of interference in the operation of the magnetometer 130 by magnetic fields (e.g., fringe fields) produced by the magnetic flux source 135 is below some specified level. For example, the distance between the magnetic flux source 135 and the magnetometer 130 could be specified to minimize a degree of magnetic field inhomogeneity in the blood vessel 150 proximate the magnetometer 130 (e.g., in examples wherein the magnetometer 130 is configured to detect magnetic fields produced by the magnetized nanoparticles 160 by detecting precession frequencies of atomic nuclei proximate the magnetized nanoparticles 160 using nuclear magnetic resonance). Additionally or alternatively, the distance between the magnetic flux source 135 and the magnetometer 130 could be specified to be less than a specified maximum distance such that a specified amount of nanoparticles 160 that are magnetized by the magnetic flux source 135 and subsequently flow downstream to be proximate the magnetometer 130 are still magnetized when they flow proximate the magnetometer 130. Such a maximum distance could be related to a magnetic relaxation time of the nanoparticles, a flow rate of blood in the blood vessel 150, or some other factors.

The analyte 170 and nanoparticles 160 are configured and distributed in the blood vessel 150 such that multiple nanoparticles 160 can bind to a single instance of the analyte 170 (e.g., to a single cancer cell). Further, nanoparticles 160 that are not bound to the analyte 170 are generally singly distributed throughout the blood in the blood vessel 150. As a result, the existence of an aggregate of nanoparticles 160 located proximate to each other could be related to the presence of one or more instances of the analyte 170 proximate the aggregate of nanoparticles. Additionally or alternatively, the velocity, angular velocity, magnetic properties (e.g., magnetic moment, coercivity, type of magnetic behavior (e.g., ferromagnetism, paramagnetism, superparamagnetism)), or other properties of the nanoparticles 160 could be related to binding to the analyte 170 and/or to some other properties of the analyte 170, nanoparticles 160, and/or the blood vessel 150.

The magnetometer 130 could be configured to detect the magnitude, direction, magnitude parallel to a specified direction, frequency, rate of change, or other properties of the magnetic field at a particular location. The particular location could be a location on or within the magnetometer. The particular location could be a volume of space within the magnetometer, e.g., the magnetometer could be configured to detect the average magnitude of the magnetic field across a sensing volume within the magnetometer (e.g., a sensing volume that contains a high-temperature, high-density gas of alkali metal atoms that is optically interrogated by the magnetometer).

The magnetometer could be configured and/or operated to detect a magnitude or other properties of a time-varying magnetic field within a specified range of frequencies (e.g., the magnetometer 130 could be a radio frequency atomic magnetometer). For example, the magnetometer 130 could be tuned to detect time-varying magnetic fields at frequencies approximately equal to a harmonic of a time-varying magnetic field to which the nanoparticles are exposed (e.g., a time-varying magnetic field produced in an environment proximate the magnetometer 130). In some examples, the magnetometer 130 could be configured to rotate magnetic spins of atomic nuclei (e.g., using one or more pulse emitters configured to emit pulses of electromagnetic radiation at the Larmor frequency of the atomic nuclei) and to detect magnetic and/or electromagnetic fields emitted by the rotated magnetic spins of the atomic nuclei as they responsively precess. The magnetometer 130 could be configured to detect the magnetic field with a specified sensitivity such that the magnetometer 130 can detect magnetic fields produced by magnetized nanoparticles 160 proximate the magnetometer (e.g., magnetized nanoparticles located less than approximately 1 centimeter from a sensing volume of the magnetometer 130). For example, the magnetometer could have a sensitivity that is less than approximately 10 femtoteslas.

FIG. 1B illustrates an example signal 131 detected by the magnetometer 130 over time. The signal 131 represents a property of a magnetic field detected by the magnetometer 130 and/or a property or variable determined therefrom. For example, signal 131 could represent the magnitude of the detected magnetic field over time. In another example, the signal 131 could represent the amplitude of the detected magnetic field at a specified frequency (e.g., a harmonic of a frequency of a magnetic field emitted by the device 100 to excite the magnetized nanoparticles 160). In a further example, the signal 131 could be related to a nuclear magnetic time constant determined from magnetic or electromagnetic fields detected by the magnetometer 130 (e.g., a T1, T2, T2*, or other time constant related to the behavior of the magnetic moments of atomic nuclei proximate the magnetometer 130). The signal 131 is intended to represent any detected or determined property of a magnetic field that could be detected by a magnetometer as described herein and that is related to an amount of magnetized nanoparticles proximate such a magnetometer.

As shown in FIG. 1B, the signal 131 includes a number of pulses 133a, 133b related to respective increases in the signal that is related to and/or determined from one or more properties of the magnetic field detected by the magnetometer 130. These pulses are related to the flow of blood 155 in the blood vessel 150 causing one or more magnetized nanoparticles 160 (e.g., single magnetized nanoparticles, aggregates of magnetized nanoparticles bound to the analyte 170) to become proximate to the magnetometer 130 (e.g., to become sufficiently proximate that the magnetic field produced by the one or more magnetized nanoparticles and or an effect thereof can be detected by the magnetometer 130) and subsequently to move away from the magnetometer 130.

The signal 131 includes lower-amplitude pulses 133b corresponding to the motion of individual magnetized nanoparticles 160 (e.g., magnetized nanoparticles that are not bound to the analyte 170) through the blood vessel 150 proximate the magnetometer 130. The signal 131 additionally includes higher-magnitude pulses 133a corresponding to the motion of aggregates of magnetized nanoparticles 160 (e.g., the aggregates may include magnetized nanoparticles bound to the analyte 170) through the blood vessel 150 proximate the magnetometer 130. The body-mountable device 100 could determine and/or detect the presence or other properties of the analyte 170 and/or of the magnetized nanoparticles 160 in the blood vessel 150 based on the width, amplitude, timing, or other properties of the detected pulses 133a, 133b. For example, a number of magnetized nanoparticles 160 proximate the magnetometer 130 at a particular time corresponding to a particular pulse detected in the signal 131 could be determined based on the amplitude of the particular pulse. For example, it could be determined that a single magnetized nanoparticle 160 is proximate to the magnetometer 130 at points in time corresponding to the lower-amplitude pulses 133b and that a plurality of magnetized nanoparticles 160 are proximate to the magnetometer 130 at points in time corresponding to the higher-amplitude pulses 133a. Related to this, it could be determined that an instance of the analyte 170 (e.g., a cancer cell) is proximate to the magnetometer at particular points in time corresponding to the higher-amplitude pulses 133a (e.g., related to the aggregation of the magnetized nanoparticles 160 by the analyte 170 causing an increase in the amplitude of the detected magnetic field).

Further, a size, number, or other properties of the analyte 170 could be determined based on the amplitude, width, shape, or other properties of the higher-amplitude pulses 133a and/or based on some other property of a signal that is related to and/or determined from one or more properties of the magnetic field that is detected by the magnetometer 130. For example, an amplitude of a pulse in the signal 131 (e.g., an amplitude of a pulse in a detected magnetic field magnitude signal) could be related to a surface area of an instance of the analyte 170 (e.g., a greater surface area could permit more magnetized nanoparticles 160 to bind to the instance of analyte 170), a number of available nanoparticle-binding sites of the analyte 170, and/or a number of instances of the analyte 170. A amount of the analyte 170 (e.g., a concentration of the analyte, a number of instances of the analyte) in a body could be determined based on a rate of detection of instances of the analyte (e.g., a rate of higher-amplitude pulses in the signal 131), a mass flow rate of blood in the blood vessel 150, and/or other factors. A velocity of the analyte 170 and/or magnetized nanoparticles 160 could be related to a width of pulses in the signal 131. Other properties of the analyte 170, the magnetized nanoparticles 160, the blood vessel 150, and/or the arm 190 could be detected and/or determined based on other features of a signal that is related to and/or determined from one or more properties of a magnetic field that is detected by a magnetometer 130.

The signal 131 could represent the magnitude of the magnetic field detected by the magnetometer 130, the magnitude of the detected magnetic field in a particular direction, the amplitude or intensity of a time-varying (e.g., oscillating) magnetic field, the amplitude or intensity of a time-varying magnetic field within a range of frequencies, a time constant (e.g., T1, T2, T2*) or other property of magnetic spins of atomic nuclei proximate the magnetometer 130 that is detected or determined from a magnetic field detected by the magnetometer 130, or some other detected and/or determined property of a magnetic field detected by the magnetometer 130. Further, a detected and/or determined property of the detected magnetic field over time could be similar or different from the illustrated example signal 131. Binding of the magnetized nanoparticles 160 to instances of the analyte 170 could be determined and/or detected based on other detected properties of the magnetic field detected by the magnetometer 130 and/or by additional or alternative features thereof. For example, a velocity, an angular velocity, or some other property of motion of one or more magnetized nanoparticles 160 could be related to whether the magnetized nanoparticle is bound to one or more instances of the analyte 170. That is, magnetized nanoparticles 160 bound to the analyte 170 could be hindered from rotating by the analyte 170, could be sped or slowed in the flow 155 of blood in the blood vessel 150 by the analyte 170 (e.g., due to a drag coefficient of the analyte 170), or could exhibit some other property or behavior that is related to binding to the analyte 170 and that can be detected using the magnetometer 130.

Nanoparticles could be magnetized by a magnetic flux source (e.g., 135) that is proximate to a magnetometer (e.g., 130) that is configured to detect a magnetic field produced by and/or related to such magnetized nanoparticles. In such examples, the magnetic flux source could act to produce a magnetizing flux during a first period of time and to produce less magnetic flux (e.g., to produce substantially no magnetic flux) during a second period of time. For example, the magnetic flux source could be operated in such a way to reduce a magnitude of an interfering magnetic field (e.g., a magnitude of fringe fields produced by the magnetic flux source at the location of the magnetometer, a degree of inhomogeneity in the background magnetic field proximate the magnetometer) produced by the magnetic flux source, e.g., to permit the magnetometer to more accurately detect fields related to magnetic fields produced by the magnetized nanoparticles. In some examples, this could include reducing a current applied to an electromagnet of the magnetic flux source. Additionally or alternatively, this could include mechanically actuating one or more elements of the magnetic flux source, e.g., to rotate a permanent magnet, to move a magnetic shim, to move the magnetic flux source away from the magnetometer, or to actuate one or more elements of the magnetic flux source and/or magnetometer in some other way.

Further, a magnitude or other properties of a magnetic field produced by such a magnetic flux source could be controlled over time (e.g., according to a square wave or some other time-varying pattern or waveform) to increase an accuracy or to otherwise improve the detection of properties of the nanoparticles and/or an analyte bound thereto based on magnetic fields detected using the magnetometer. For example, a magnetic flux source could produce a magnetic field having a magnitude that varies according to a square wave having a frequency of approximately 10 Hertz. In such an example, a magnetic field that is related to properties of the nanoparticles (e.g., a magnitude of the magnetic field produced by nanoparticles proximate the magnetometer, an amplitude of a time-varying magnetic field produced by the nanoparticles in response to exposure to an exciting time-varying magnetic field) could be detected by the magnetometer and demodulated or otherwise operated on based on the frequency of the time-varying magnetic field produced by the magnetic flux source.

Note that the use of the magnetometer 130 to detect magnetic fields produced by magnetized nanoparticles 160 in a flow 155 of blood in a blood vessel 150 that have been magnetized by an upstream magnetic flux source 135 and further to determine properties of the magnetized nanoparticles 160 and/or an analyte 170 to which the nanoparticles 160 are configured to bind is intended as a non-limiting illustrative example of embodiments described herein. Nanoparticles could be disposed in a variety of different environments (e.g., other bodily fluids, fluids of an animal, fluids of a natural environment, fluids of a medical, scientific, or industrial process). The embodiments herein could be applied to the detection and/or determination of properties of magnetized nanoparticles and/or analytes in an ex vivo and/or in vitro flow cytometry experiment or process. One or more magnetometers configured to detect magnetic fields produced by magnetized nanoparticles could be disposed in a wearable, body-mountable, handheld, desktop, floor-, wall-, ceiling-, or otherwise-mounted, or otherwise configured device or system. Further, methods and systems described herein could be used with permanently magnetic and/or magnetized nanoparticles. The nanoparticles could be disposed in a flowing fluid or in a substantially static fluid according to an application. Other environments and applications are anticipated.

Nanoparticles as described herein are configured to be magnetized by an externally applied magnetic field (e.g., a magnetic field produced by a magnetic flux source of a wearable device as described herein). Such nanoparticles could additionally be configured to become less magnetized over time, e.g., such that the nanoparticles do no remain magnetized and aggregate, forming aggregates that could have negative health effects (e.g., that could block blood vessels) and/or that could have negative effects on applications of the nanoparticles (e.g., that could reduce a degree of interaction of the nanoparticles with an analyte of interest and/or that could produce false positives in embodiments wherein the nanoparticles are configured to aggregate by binding to the analyte of interest). In such examples, the time-dependence of the magnetization of the nanoparticles could be related to a magnetic relaxation time of the nanoparticles. Such a magnetic relaxation time could be related to a time constant of an exponential or otherwise time-dependent function related to the decrease of the magnetization of a magnetized nanoparticle over time.

Additionally or alternatively, such a magnetic relaxation time could be related to a time-dependence of a statistical process of the magnetized nanoparticles. For example, the magnetic relaxation time could be related to an expected time during which a magnetic property of a magnetized nanoparticle changes (e.g., a mean time between the instances of the magnetic moment of a nanoparticle flipping direction, changing orientation, becoming demagnetized, or otherwise changing from a first state to a second state). For example, the nanoparticles could each include one or more particles of superparamagnetic material (e.g., superparamagnetic iron oxide) and the magnetic relaxation time of the nanoparticles could be related to the Neel relaxation time of the particles of superparamagnetic material of the nanoparticles.

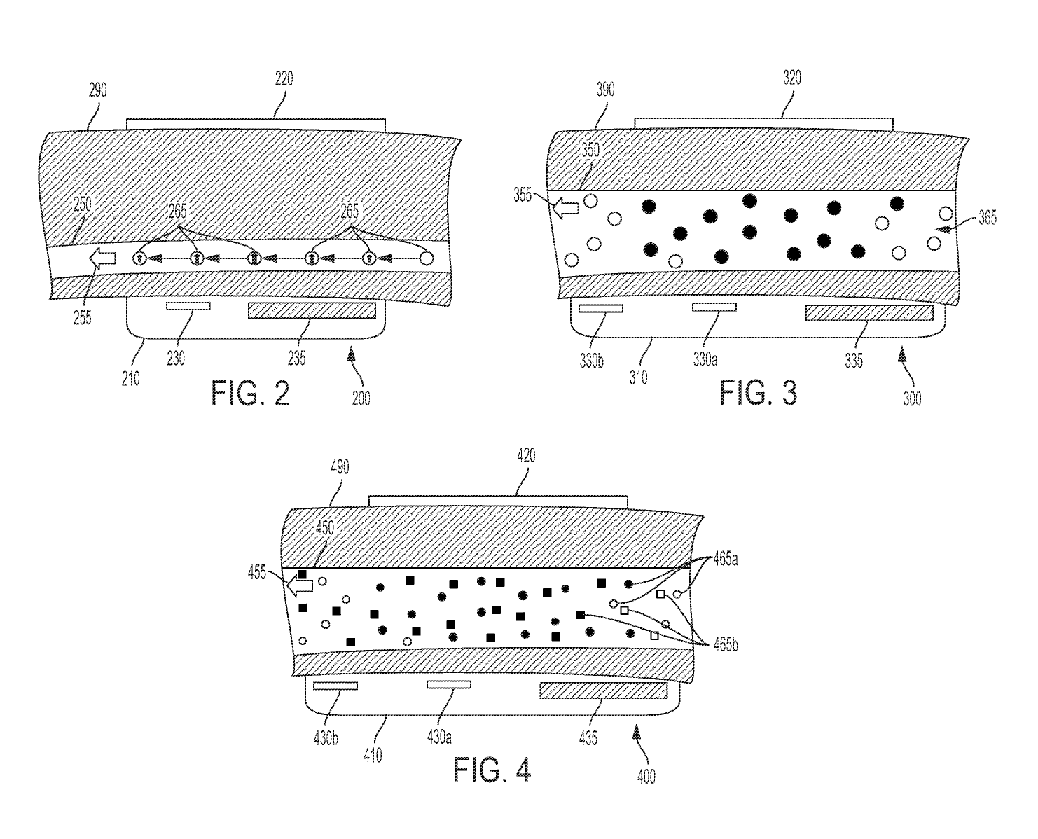

To illustrate the operation of such systems and/or devices to magnetize nanoparticles and subsequently detect magnetic fields related to such magnetized nanoparticles, FIG. 2 illustrates an example nanoparticle complex 265 that is disposed in a blood vessel 250 (i.e., a portion of subsurface vasculature). The complex 265 includes one or more nanoparticles that are bound to an analyte. The blood vessel 250 is located in an arm 290 and contains blood that is flowing (direction of flow indicated by the arrow 255). FIG. 2 illustrates the motion of the complex 265 in the blood vessel 250 over time in the direction of the flow 255. Arrows in the illustrated complex 265 over time indicate the degree of magnetization of the complex 265 over time. A body-mountable device 200 includes a housing 210 mounted outside of the blood vessel 250 by a mount 220 configured to encircle the arm 290. The body-mountable device 200 includes a magnetometer 230 disposed in the housing 210 and configured to detect magnetic fields at a location outside of the arm 290 (e.g., at a location within the magnetometer 230). The body-mountable device 200 additionally includes a magnetic flux source 235 (e.g., a permanent magnet, an electromagnet) disposed in the housing 210 and configured to produce a magnetic flux and/or field sufficient to at least partially magnetize and/or align a magnetic dipole of the one or more nanoparticles of the complex 265. For example, the magnetic flux source 235 could be configured to produce a magnetic field in the blood vessel 250 that has a strength greater than approximately 100 Gauss.

As shown in FIG. 2, the complex 265 is moved by the blood flow 255 past the magnetic flux source 235. This can result in the nanoparticle(s) of the complex 265 becoming and/or being magnetized (illustrated by the increasing size of the arrows as the complex 265 passes over the magnetic flux source 235). The magnetometer 230 can then detect a magnetic field produced by the magnetized nanoparticle(s) of the complex 265 and/or a magnetic field related to such a produced magnetic field (e.g., a time-varying magnetic field produced by precessing magnetic spins of atomic nuclei in the blood vessel 250 that are precessing at a frequency related to the magnetic field produced by the magnetized complex 265). The detected magnetic field could be used to determine one or more properties of the magnetized complex 265 (e.g., properties of the one or more nanoparticles thereof), the analyte, and/or the environment (e.g., the blood in the blood vessel 250). For example, a rate of reduction of the magnetization of the magnetized nanoparticles, a rate of rotation of the magnetized nanoparticles (in examples wherein the nanoparticles are aligned by the magnetic field produced by the magnetic flux source 235), a degree of aggregation of the magnetized nanoparticles, or some other detected properties of the magnetized nanoparticles and/or the complex 265 could be detected and/or determined.

Note that, as illustrated in FIG. 2, the degree of magnetization of the complex 265 (e.g., of a nanoparticle of the complex) is represented by a gradually increasing and gradually decreasing quantity. Such a continuously-valued magnetization could be related to an orientation of the magnetic moment of one or more particles of magnetic material and/or one or more magnetic domains thereof. Such a continuously-valued magnetization could additionally or alternatively be related to a degree of magnetization of a particle of magnetic material that includes more than one magnetic domain before being magnetized. Alternatively, the magnetization of the complex 265 could be a discrete-valued property. For example, the magnetization of a particular nanoparticle and/or complex of nanoparticles transitions from a first state (e.g., an un-magnetized state, a random state of a discrete set of states, e.g., parallel and antiparallel spin states) to a second state (e.g., a magnetized state parallel to a field produced by the magnetic flux source 235) in response to exposure to a magnetic field generated by the magnetic flux source 235. The magnetization of the particular nanoparticle and/or complex of nanoparticles could further transition to a third state (e.g., a randomly selected state) subsequent to magnetization (e.g., subsequent to passing the magnetometer 230).

In some examples, magnetization of the particular nanoparticle and/or complex of nanoparticles could include changing a plurality of such discrete-valued or continuous-valued magnetic states of respective nanoparticles and/or magnetic elements thereof (e.g., particles of superparamagnetic iron oxide). For example, magnetization could include aligning the discrete-valued magnetizations of a plurality of nanoparticles and/or magnetic elements thereof such that the nanoparticle and/or complex 265 produces a greater external magnetic field (e.g., due to the magnetic fields produced by the individual nanoparticles and/or elements thereof being aligned such that they sum rather than partially cancel). Such a nanoparticle and/or complex of nanoparticles becoming less magnetized over time could include the magnetizations of the plurality of nanoparticles and/or magnetic elements thereof becoming less aligned such that the nanoparticle and/or complex 265 produces a lesser external magnetic field (e.g., due to the magnetic fields produced by the individual nanoparticles and/or elements thereof not being aligned).

A distance between the magnetic flux source 235 and the magnetometer 230 configured to detect magnetic fields produced by nanoparticles magnetized by the magnetic flux source 235, a length and strength of the magnetic flux source 235, and other properties of devices and systems as described herein could be related to the magnetic relaxation time of the nanoparticles, among other factors (e.g., the flow rate of a fluid, e.g., blood, carrying the magnetized nanoparticles from the magnetic flux source to the magnetometer). The magnetic relaxation time of the nanoparticles could be specified such that the nanoparticles substantially do not aggregate when not magnetized and/or not in the presence of an instance of an analyte to which the nanoparticles are configured to bind, e.g., the magnetic relaxation time of the nanoparticles could be less than some specified value (e.g., less than between approximately 1 second and approximately 2 seconds).

The distance between the magnetic flux source 235 and the magnetometer 230 could be greater than some minimum distance to reduce an interference in the operation of the magnetometer 230 by magnetic fields produced by the magnetic flux source 235 (e.g., fringe field produced by the magnetic flux source 235 that are detect by and/affect a magnetic field detected by the magnetometer 230). For example, the magnetic flux source 235 and magnetometer 230 could be separated by a distance on the order of a few centimeters (e.g., greater than approximately 1 centimeter). As a result, the magnetic relaxation time of the nanoparticles could be greater than some specified minimum time such that an amount of the nanoparticles magnetized by the magnetic flux source are still magnetized when they have travelled (e.g., been carried by blood flow in the blood vessel 250) downstream to a location proximate the magnetometer 230. Such a minimum relaxation time could be related to the distance between the magnetometer 230 and the magnetic flux source 235 and the flow velocity of blood in the blood vessel 250. For example, the magnetic relaxation time of the nanoparticles could be greater than between approximately 100 milliseconds and approximately 1 second.

In some examples, nanoparticles used in combination with systems, devices, and methods as described herein (e.g., systems including a magnetic flux source configured to magnetize such nanoparticles and magnetometers to detect magnetic fields related to such magnetized nanoparticles) could have magnetic relaxation times within a specified narrow range of relaxation times. Detection of one or more properties of such nanoparticles and/or of analytes to which such nanoparticles are configured to bind could be related to such a specified narrow range of magnetic relaxation times of the nanoparticles. For example, a system could include two or more magnetometers disposed at respective locations along a blood vessel. Such a system could be operated to detect magnetic fields at the respective different locations and to determine improved (e.g., higher accuracy, lower noise) estimates of properties of the nanoparticles and/or an analyte bound thereto based on magnetic field detected at the different locations by the magnetometers.

FIG. 3 illustrates example nanoparticle complexes 365 that are disposed in a blood vessel 350 (i.e., a portion of subsurface vasculature). The complexes 365 each include one or more nanoparticles. The blood vessel 350 is located in an arm 390 and contains blood that is flowing (direction of flow indicated by the arrow 355). FIG. 3 illustrates the motion of the complexes 365 in the blood vessel 350 over time in the direction of the flow 355. The fill color in the illustrated complexes 365 indicate the degree of magnetization of each complex 365; a black-filled complex is magnetized while a white-filled complex is substantially not magnetized. Note that magnetization of the complexes 365 is illustrated at a discrete, binary state for illustration purposes only; the magnetization of the complexes and/or nanoparticles thereof could be continuous-valued or discrete and having a number of possible values greater than 2. A body-mountable device 300 includes a housing 310 mounted outside of the blood vessel 350 by a mount 320 configured to encircle the arm 390. The body-mountable device 300 includes a first 330a and second 330b magnetometers disposed in the housing 310 and configured to detect magnetic fields at respective first and second locations outside of the arm 390 (e.g., at locations within the magnetometers 330a, 330b). The body-mountable device 300 additionally includes a magnetic flux source 335 (e.g., a permanent magnet, an electromagnet) disposed in the housing 310 and configured to produce a magnetic flux and/or field sufficient to at least partially magnetize and/or align a magnetic dipole of the one or more nanoparticles of the complexes 365.

As shown in FIG. 3, the complexes 365 are moved by the blood flow 355 past the magnetic flux source 335. This can result in the nanoparticle(s) of the complexes 365 becoming and/or being magnetized (illustrated by the complexes 365 being more likely to be black-filled, i.e., magnetized, as the complexes 365 pass over the magnetic flux source 335). The magnetometers 330a, 330b can then detect magnetic fields related to nanoparticle(s) of the complexes 365. As shown in FIG. 3, the first magnetometer 330a is disposed proximate a first portion of subsurface vasculature wherein nanoparticles of the complexes 365 that were magnetized by the magnetic flux source 335 are substantially still magnetized (e.g., a location that is, based on a magnetic relaxation time of the nanoparticles and a flow rate of blood in the blood vessel 350, sufficiently close to the magnetic flux source). The second magnetometer 330b is disposed proximate a second portion of subsurface vasculature that is downstream from the first portion such that nanoparticles of the complexes 365 that were magnetized by the magnetic flux source 335 have become less magnetized and/or have become un-magnetized. A more accurate or otherwise improved estimate of a property of the complexes 365, nanoparticle(s) thereof, and/or an analyte bound thereto could be determined based on the signals generated by the first 330a and second 330b magnetometers. For example, a signal (e.g., a detected magnetic field, a determined magnetic resonance time constant) produced by the second magnetometer 330b could be used as a background signal (i.e., a signal corresponding to no nanoparticles and/or non-magnetized or un-magnetized nanoparticles) for comparison with a signal produced by the first magnetometer 330a (i.e., a signal corresponding to no nanoparticles and/or magnetized nanoparticles).

The distances of the magnetometers 330a, 330b and/or additional magnetometers (not shown) of the device 300 could be set based on a range of magnetic relaxation times of the nanoparticles in the complexes 365 and on an expected flow rate of blood in the blood vessel 350. Additionally or alternatively, the system 300 could include additional magnetometers (not shown) disposed at further different distances from the magnetic flux source 335 and could use outputs generated by the additional magnetometers to perform such a determination of properties of the complexes 365, nanoparticles thereof, and/or an analyte bound thereto. In some examples, this could include detecting the flow velocity or rate of blood in the blood vessel 350 (e.g., using laser speckle velocimetry, ultrasonic velocimetry, or some other method) and using such information to determine the properties of the complexes 365, nanoparticles, and/or analyte, e.g., by determining respective sets of the magnetometers corresponding to regions of the blood vessel 350 wherein the complexes 365 are magnetized and regions wherein the complexes 365 are substantially non-magnetized.

In some examples, nanoparticles used in combination with systems, devices, and methods as described herein (e.g., systems including a magnetic flux source configured to magnetize such nanoparticles and magnetometers to detect magnetic fields related to such magnetized nanoparticles) could include multiple different sets of nanoparticles having magnetic relaxation times within respective different specified narrow ranges of relaxation times. In such examples, nanoparticles of each set could be configured to selectively interact with (e.g., bind to) respective different analytes. Detection of one or more properties of such sets of nanoparticles and/or of respective analytes to which such sets of nanoparticles are configured to bind could be related to such specified narrow ranges of magnetic relaxation times of the sets of nanoparticles.

FIG. 4 illustrates example first 465a and second 465b nanoparticle complexes that are disposed in a blood vessel 450 (i.e., a portion of subsurface vasculature). The complexes 465a, 465b each include one or more nanoparticles. The blood vessel 450 is located in an arm 490 and contains blood that is flowing (direction of flow indicated by the arrow 455). FIG. 4 illustrates the motion of the complexes 465a, 465b in the blood vessel 450 over time in the direction of the flow 455. The fill color in the illustrated complexes 465a, 465b indicate the degree of magnetization of each complex 465a, 465b; a black-filled complex is magnetized while a white-filled complex is substantially not magnetized. Note that magnetization of the complexes 465a, 465b is illustrated at a discrete, binary state for illustration purposes only; the magnetization of the complexes and/or nanoparticles thereof could be continuous-valued or discrete and having a number of possible values greater than 2. A body-mountable device 400 includes a housing 410 mounted outside of the blood vessel 450 by a mount 420 configured to encircle the arm 490. The body-mountable device 400 includes a first 430a and second 430b magnetometers disposed in the housing 410 and configured to detect magnetic fields at respective first and second locations outside of the arm 490 (e.g., at locations within the magnetometers 430a, 430b). The body-mountable device 400 additionally includes a magnetic flux source 435 (e.g., a permanent magnet, an electromagnet) disposed in the housing 410 and configured to produce a magnetic flux and/or field sufficient to at least partially magnetize and/or align a magnetic dipole of the one or more nanoparticles of the complexes 465a, 465b.

As shown in FIG. 4, the complexes 465a, 465b are moved by the blood flow 455 past the magnetic flux source 435. This can result in the nanoparticle(s) of the complexes 465a, 465b becoming and/or being magnetized (illustrated by the complexes 465a, 465b being more likely to be black-filled, i.e., magnetized, as the complexes 465a, 465b pass over the magnetic flux source 435). The magnetometers 430a, 430b can then detect magnetic fields related to nanoparticle(s) of the complexes 465a, 465b that are magnetized when such magnetized complexes move proximate to the magnetometers 430a, 430b. The first 465a and second 465b complexes have respective different magnetic relaxation times (e.g., nanoparticles of the complexes have magnetic relaxation times within respective different ranges of relaxation times). Specifically, the first set of complexes 465a has a shorter magnetic relaxation time than the second set of complexes 465b.

This is shown in FIG. 4, wherein the first magnetometer 430a is disposed proximate a first portion of subsurface vasculature wherein nanoparticles of both sets of complexes 465a, 465b that were magnetized by the magnetic flux source 435 are substantially still magnetized. The second magnetometer 430b is disposed proximate a second portion of subsurface vasculature that is downstream from the first portion such that nanoparticles of the first set of complexes 465a that were magnetized by the magnetic flux source 435 have become less magnetized and/or have become un-magnetized. Conversely, the second portion of subsurface vasculature is located such that nanoparticles of the second set of complexes 465b that were magnetized by the magnetic flux source 435 are substantially still magnetized. An estimate of a property of both sets of complexes 465a, 465b, nanoparticle(s) thereof, and/or the respective analytes bound thereto could be determined based on the signals generated by the first 430a and second 430b magnetometers.

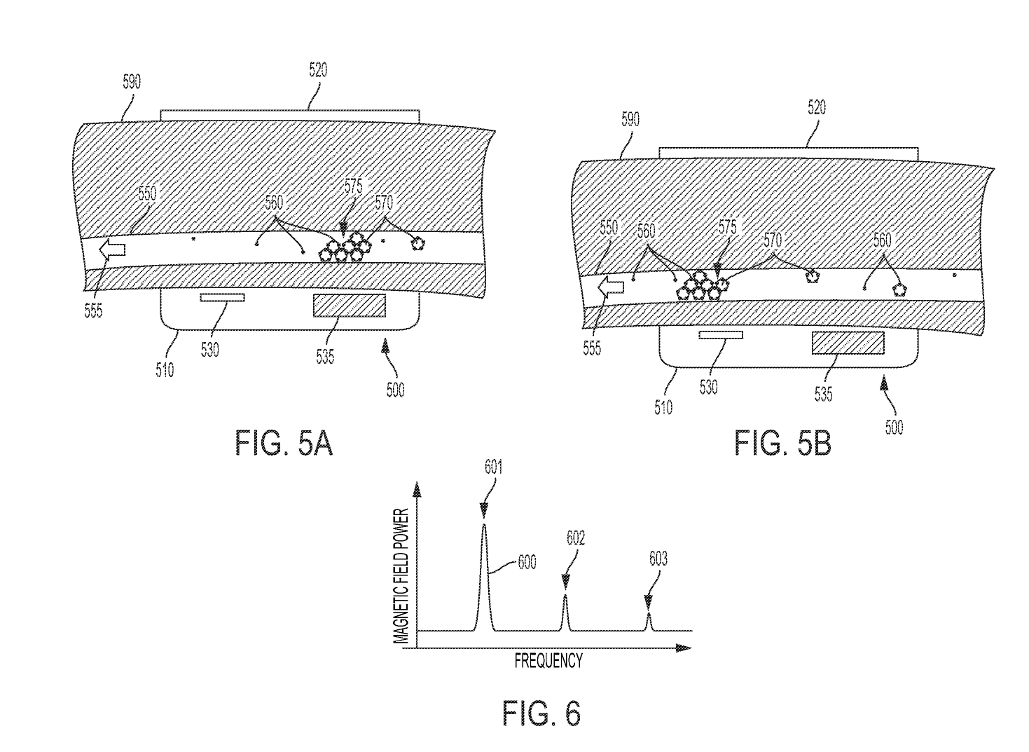

In some examples, magnetized nanoparticles and/or analytes bound to such magnetized nanoparticles in an environment could be collected such that a magnitude of the magnetic field produced by the magnetized particles and detected by a magnetometer is increased, e.g., to improve a determination of a property of the analyte by, e.g., increasing a magnitude of the detected magnetic field. FIGS. 5A and 5B illustrate, during respective first and second periods of time, example magnetized nanoparticles 560 and an analyte of interest 570 with which the nanoparticles 570 are configured to selectively interact disposed in a blood vessel 550 (i.e., a portion of subsurface vasculature). The blood vessel 550 is located in an arm 590 and contains blood that is flowing (direction of flow indicated by the arrow 555). A body-mountable device 500 includes a housing 510 mounted outside of the blood vessel 550 by a mount 520 configured to encircle the arm 590. The body-mountable device 500 includes a magnetometer 530 disposed in the housing 510 and configured to detect a magnetic field at a location outside of the arm 590 (e.g., at a location within the magnetometer 530).

The body-mountable device 500 additionally includes a magnetic flux source 535 (e.g., a permanent magnet, an electromagnet) configured to magnetize the nanoparticles 560 and to exert an attractive magnetic force on the magnetized nanoparticles 560 such that at least some of the magnetized nanoparticles 570 in the blood vessel 550 are collected proximate the magnetic flux source 535. Such a magnetic flux source could be considered a collection magnet. In the example shown in FIGS. 5A and 5B, this includes collecting magnetized nanoparticles 560 that are bound to instances of the analyte 570 into a bolus 575 located proximate the magnetic flux source 535. Note that, in some examples, separate components (e.g., separate permanent magnets) of the device 500 could be configured to, respectively, magnetize the nanoparticles and to collect the magnetized nanoparticles.

FIG. 5A shows the body-mountable device 500 during a first period of time during which the magnetic flux source 535 is exerting an attractive magnetic force to attract magnetized nanoparticles 560 and instances of the analyte 570 bound thereto to form a bolus 575 of collected magnetized nanoparticles 560. FIG. 5B shows the body-mountable device 500 during a second period of time. The magnetic flux source 535 is configured and/or operated during the second period of time to exert a lesser magnetic force (e.g., to exert substantially no magnetic force) on the magnetized nanoparticles 560 such that the bolus 575 is released from the proximity of the magnetic flux source 535 and flows within the blood vessel 550 to a downstream location, past the magnetometer 530. The magnetometer 530 operates to detect a magnetic field produced by the magnetized nanoparticles 560 and/or a magnetic field related to such a produced magnetic field (e.g., by magnetized nanoparticles of the bolus 575) to determine a property of the magnetized nanoparticles 560, the analyte 570, and/or the bolus 575. For example, a number of instances of the analyte 570 in the bolus 575 (and/or a concentration or number of the analyte 570 in the blood overall) could be determined based on a magnitude, nuclear magnetic time constant, or other properties of and/or determined form the detected magnetic field.

Note that the configuration and operation shown in FIGS. 5A and 5B are non-limiting examples. In some embodiments, a magnetic flux source could be co-located with a magnetometer (e.g., could act to collect and/or magnetize nanoparticles proximate the magnetometer). In some examples, the magnetometer could operate to detect the magnetic field produced by the magnetized nanoparticles while the magnetic flux source is exerting an attractive magnetic force to collect the magnetized nanoparticles (e.g., by introducing a bias magnetic field using a coil or other magnetic materials to cancel the magnetic field generated by the magnetic flux source that is detected by the magnetometer, by configuring the magnetometer to detect magnetic fields in a direction perpendicular to a field produced by a magnetic flux source, by detecting oscillating magnetic fields produced by the magnetized nanoparticles, e.g., in response to exposure to an oscillating magnetic field produced by an excitation coil).

Magnetometers, devices containing magnetometers, nanoparticles, and other aspects and embodiments described herein (e.g., 100, 200, 300, 400, 500) could be configured and/or operated to provide a variety of applications. In some examples, nanoparticles could be configured to bind to an analyte of interest, and one or more magnetometers could detect a magnetic field produced by the magnetized nanoparticles to determine one or more properties (e.g., a presence, a location, a number, a concentration) of the analyte. In some examples, a device could be configured to magnetize, collect, release, separate, modify, or otherwise manipulate the magnetized nanoparticles to enable the detection, extraction, modification, or other manipulation of the analyte. Additionally or alternatively, the system could include an energy emitter and the energy emitter could emit energy toward collected magnetized nanoparticles and/or when it is detected that the analyte is present to alter one or more properties of the analyte (e.g., to destroy, denature, heat, change a conformation state of, other otherwise modify the analyte). In some examples, detection of one or more properties of an analyte bound to magnetized nanoparticles could enable the determination of a course of medical treatment, the adjustment of a dosage of a drug, the generation of a medical alert, or some other action. Other configurations, operations, and applications of the embodiments described herein are anticipated.

The terms “binding”, “bound”, and related terms used herein are to be understood in their broadest sense to include any interaction between the receptor and the target or another functionalized particle such that the interaction allows the target to be modified or destroyed by energy emitted from a wearable device.

### III. EXAMPLE METHODS FOR DETECTING NANOPARTICLES

Magnetometers of embodiments described herein could be configured to detect magnetic fields produced intrinsically by nanoparticles, e.g., produced by permanently and/or spontaneously magnetic elements of the nanoparticles. Additionally or alternatively, the nanoparticles could be induced to produce a magnetic field, e.g., by being temporarily or permanently magnetized, by being exposed to an oscillating or otherwise time-varying electromagnetic field, or by some other means. Additionally or alternatively, magnetometers could be configured to detect magnetic fields related to magnetic fields produced by magnetized nanoparticles. For example, precessing magnetic spins of atomic nuclei (e.g., hydrogen atoms in water) could precess at a frequency that is related to the magnetic field magnitude in the environment of the atomic nuclei, e.g., related to a magnetic field that is a combination of the Earth's magnetic field and a magnetic field produced by magnetized nanoparticles. Such magnetic spins of atomic nuclei could produce time-varying magnetic fields related to the precession and the magnetometer could detect such produced magnetic fields (e.g., the magnetometer could be configured to indirectly detect magnetic fields produced by magnetized nanoparticles by using the techniques of nuclear magnetic resonance to detect the effects of such magnetized nanoparticles on proximate atomic nuclei).

A magnetometer could be configured to directly detect the magnetic field produced by one or more magnetized nanoparticles. The direction, magnitude, and/or other properties of the produced magnetic field at a particular location can be related to the location and/or orientation of the one or more magnetized nanoparticles relative to the particular location, the magnitude of the permanent and/or induced magnetic dipole moment of the magnetized nanoparticles, magnetic properties of materials proximate the particular location, or other factors. A magnetic field at the particular location (e.g., a direction and/or magnitude of a magnetic field detected by, e.g., a magnetometer) could be related to the magnetic field of the earth, magnetic fields produced by electronics or other devices proximate the particular location, magnetic and/or electromagnetic fields produced by atomic magnetic spins that are precessing in a magnetic field (e.g., a magnetic field produced by the Earth and/or by one or more magnetized nanoparticles), magnetized or otherwise magnetic materials proximate the particular location, or other factors in addition to the magnetic field produced by the one or more magnetized nanoparticles.

In such examples wherein the magnetometer is configured to directly detect magnetic fields produced by magnetized nanoparticles, the magnetometer could have a sensitivity below approximately 10 femtoteslas to, e.g., permit detection of magnetic fields produced by the magnetized nanoparticles that are within an environment of interest that is displaced from the magnetometer by some distance, e.g., magnetized nanoparticles that are disposed in a portion of subsurface vasculature that is approximately 1 centimeter from the magnetometer beneath a skin surface to which the magnetometer is mounted. Such a magnetometer could be an optical atomic magnetometer, i.e., a magnetometer configured to detect magnetic fields by optically pumping and/or optically detecting a state of atoms in a gas (e.g., atoms of a metal vapor comprising cesium, rubidium, potassium, or some other fermionic atoms) that is related to the magnitude, direction, magnitude in a specified direction, or some other property of the magnetic field within the gas. For example, the magnetometer could be a spin-exchange relaxation-free (SERF) magnetometer configured to detect low-frequency components of a magnetic field in one or more specified directions. Additionally or alternatively, the magnetometer could include a multipass scalar atomic magnetometer configured to detect the magnitude of the magnetic field.

In some examples, a magnetometer could detect the response of magnetized nanoparticles to a provided external energy, e.g., to an oscillating or otherwise time-varying magnetic field produced in an environment of interest (e.g., produced by one or more pulse emitters and/or excitation coils). This could include detecting magnetic fields produced by the magnetized nanoparticles in response to such provided external energy, e.g., detecting a time-varying magnetic field produced by magnetized nanoparticles in response to being exposed to an oscillating magnetic field. For example, a difference between a phase, frequency, magnitude, or other properties of the provided and a responsively produced oscillating magnetic fields could be used to determine a susceptibility, coercivity, degree of magnetization, degree of aggregation, or other magnetic properties of magnetized nanoparticles in an environment.

In some examples, a system could include an excitation coil (or some other antenna or other type of electromagnetic-field-producing element(s)) configured to produce an oscillating magnetic field in an environment of interest (e.g., in a portion of subsurface vasculature). The produced oscillating magnetic field could cause magnetized nanoparticles and/or other magnetic objects or materials in the environment of interest to produce a magnetic field that could be detected by a magnetometer positioned proximate to the environment of interest. One or more properties of the magnetized nanoparticles, analytes with which the magnetized nanoparticles are configured to selectively interact, and/or some other contents of the environment could be detected and/or determined based on the detected magnetic field. The magnetic field produced by the magnetized nanoparticles could include a reflected, phase-shifted, frequency-shifted, frequency-multiplied, or otherwise modified version of the field produced by the excitation coil.

For example, the magnetic field produced by the magnetized nanoparticles could include a fundamental frequency at the frequency of the oscillating field produced by the excitation coil and a number of harmonics at frequencies that are multiples of the frequency of the oscillating field. In some examples, the magnetization of the magnetized nanoparticles (e.g., the degree to which a magnetic flux source is configured and/or operated to magnetize the nanoparticles) could be specified to maximize the magnitude of such a responsively produced oscillating (or otherwise time-varying) signal. For example, the magnitude of oscillating time-varying magnetic fields that are harmonics of an exciting oscillating magnetic field and that are produced by a magnetized nanoparticle in response to exposure to the exciting field could be maximized by magnetizing the magnetized nanoparticles such that their degree of magnetization is near a particularly nonlinear aspect of a magnetization curve of the nanoparticles.

FIG. 6 shows an example power spectrum 600 of a magnetic field produced by magnetized nanoparticles in such a scenario. The magnetic field produced by the magnetized nanoparticles in response to the oscillating magnetic field produced by the excitation coil includes an oscillating field at substantially the same frequency as the frequency of the oscillating field produced by the excitation coil (the fundamental peak 601 of the power spectrum 600) and oscillating fields at multiples of the frequency of the oscillating field produced by the excitation coil (the harmonic peaks 602, 603 of the power spectrum 600). The presence, location, number, or other properties of magnetized nanoparticles proximate the magnetometer could be determined based on the amplitude, presence, phase shift, width, center frequency, or other properties of the harmonic peaks 602, 603, fundamental peak 601, and/or the aspects of the detected magnetic field corresponding to those peaks. In some examples, a filter or other means could be used to remove the fundamental peak 601 from the detected magnetic field to, e.g., increase a sensitivity of a detector to properties of the harmonic peaks 602, 603. Additionally or alternatively, the magnetometer could be tuned to detect specific components of the magnetic field (e.g., components within specified range(s) of frequencies). For example, the magnetometer could include a radio frequency atomic magnetometer tuned to a frequency corresponding to one of the harmonic peaks 602, 603.

In some examples, an exciting, time-varying (e.g., oscillating) magnetic field could be produced to have a magnitude in a first direction, and magnetic fields responsively produced by magnetized nanoparticles could be detected in a second direction that is perpendicular to the first. For example, a magnetometer could be configured to detect the magnitude of a time-varying magnetic field in the second direction while being substantially insensitive to the magnitude of magnetic fields in the first direction. This could be performed, e.g., to reduce the interference of the exciting field on the nanoparticle-related fields detected by the magnetometer. Further, an easy axis, a direction of magnetization, or some other property of the magnetized nanoparticles could be controlled, relative to the first and second directions, to increase the magnitude of the signal detected by the magnetometer and/or to reduce an amount of the exciting magnetic field that is detected by the magnetometer. For example, a direction of the an easy axis of one or more nanoparticles and/or a direction of an induced magnetic moment of magnetized nanoparticles could be controlled (e.g., by a direction of a magnetic field produced by a magnetic flux source) to be in a direction between the first and second directions, e.g., a direction that is approximately 45 degrees from each of the first and second directions.

A magnetometer configured to detect such time-varying (e.g., oscillating) magnetic fields produced by magnetized nanoparticles could include a SERF magnetometer, a radio-frequency atomic magnetometer (e.g., a radio frequency atomic magnetometer that is configured to detect contents of a time-varying magnetic field at a frequency corresponding to a harmonic of a frequency of an exciting oscillating magnetic field), a SQUID, an inductive pickup (e.g., one or more coils of wire or otherwise-formed inductive antenna(s)), or some other time-varying magnetic field detecting means. Such a magnetometer could be sensitive to time-varying magnetic fields to a level of approximately 100 femtoteslas or less.

In some examples, magnetic fields produced by magnetized nanoparticles could be detected indirectly, e.g., the effects of the magnetized nanoparticles on elements of the environment proximate the magnetized nanoparticles could be detected. For example, a fluorophore or other element of the environment could have an optical property (e.g., a fluorescence intensity, a fluorescence lifetime) that is related to the magnitude of the magnetic field to which the fluorophore is exposed (e.g., the magnitude of a magnetic field produced by a magnetized nanoparticle proximate the fluorophore) and the optical property of the fluorophore could be detected (e.g., by illuminating the fluorophore and detecting a responsively emitted light from the fluorophore). Such a fluorophore could include one or more magnetic moieties configured to change a shape of the fluorophore or to otherwise alter the fluorophore when exposed to a magnetic field such that the optical property of the fluorophore is related to the magnetic field (e.g., to a magnetic field produced by a proximate magnetized nanoparticle).

In some examples, indirectly detecting magnetic fields produced by magnetized nanoparticles could include using the techniques of nuclear magnetic resonance and/or magnetic resonance imaging to detect the effects of the magnetized nanoparticles on the magnetic spins of atomic nuclei (e.g., hydrogen atoms in water or in other compounds) that are proximate the magnetized nanoparticles. A magnetic spin of an atomic nucleus (e.g., a fermionic atomic nucleus having half-integer overall spin, e.g., a nucleus of a hydrogen atom) could, when perturbed from an equilibrium state (e.g., from alignment with a magnetic field in the environment of the atomic nucleus), precess for a period of time until it returns to the equilibrium state. Precession could occur at a frequency related to the magnitude of the background magnetic field. Thus, when a population of atomic nuclei are perturbed in a substantially homogeneous magnetic field (e.g., in the Earth's magnetic field, in the absence of a significant source of magnetic flux), the atomic nuclei will precess at substantially the same frequency. Conversely, when inhomogeneities are present in the magnetic field (e.g., inhomogeneities related to magnetic fields produced by one or more magnetized nanoparticles), the atomic nuclei will precess at a range of difference frequencies related to the range of magnetic field strengths throughout the inhomogeneous magnetic field.

Systems and devices as described herein (e.g., devices including magnetometers configured to detect magnetic fields related to magnetized nanoparticles and/or magnetic flux sources configured to magnetize such nanoparticles) could use such properties of atomic nuclei to detect properties of magnetized nanoparticles and/or of analyte to which such nanoparticles are configured to bind. That is, such systems and devices could use techniques from nuclear magnetic resonance and/or magnetic resonance imaging to detect the magnetized nanoparticles by detecting magnetic resonance time constants of the atomic nuclei (e.g., a T1 constant, a T2 constant, a T2* constant) or some other properties of the atomic nuclei. This could include polarizing the atomic nuclei (e.g., to increase a signal strength of a magnetic field produced by precessing magnetic spins of the atomic nuclei that is detected by a magnetometer), rotating the polarized atomic nuclei (e.g., such that the polarized atomic nuclei begin to precess in the background magnetic field by, e.g., operating one or more pulse emitters to emit one or more magnetic spin rotating pulses), and detecting a time-varying magnetic and/or electromagnetic field generated by the precessing magnetic spins of the atomic nuclei.

Polarizing the magnetic spins of the atomic nuclei could include exposing the atomic nuclei to a strong magnetic field, e.g., a magnetic field having a strength on the order of one to several Tesla. In some examples, such a magnetic field could be provided by a magnetic flux source of a wearable device (e.g., a permanent magnet, electromagnet, or other element(s) of such a wearable device). In some examples, such a magnetic flux source could also be configured to magnetize nanoparticles as described elsewhere herein. In some examples, the magnetic flux source could polarize the magnetic spins of the atomic nuclei at a first location and the atomic nuclei could then flow downstream (e.g., in a blood flow) to a second location at which the magnetic spins could be rotated and/or a magnetometer could detect time-varying magnetic fields produced by such rotated, precessing magnetic spins. In some examples, the magnetic flux source could be configured such that the field produced by the magnetic flux source is substantially homogeneous proximate the magnetometer and/or pulse emitter(s) used to rotate the magnetic spins. This could include locating the magnetic flux source more than some minimum distance from the magnetometer and/or pulse emitters. Additionally or alternatively, the magnetic flux source could be operated to reduce the magnitude and/or inhomogeneity of the produced polarizing field (e.g., by reducing a current applied to an electromagnet of the magnetic flux source, by rotating or otherwise actuating a permanent magnet and/or magnetic shim of the magnetic flux source, by moving the magnetic flux source away from the magnetometer and/or pulse emitter(s)). In some examples, an electromagnet, permanent magnet, magnetic shims, or other elements could be configured and/or operated to reduce an inhomogeneity of the magnetic field proximate the magnetometer and/or pulse emitter(s).

Rotating the polarized atomic nuclei could include emitting a pulse of an oscillating magnetic field oriented in a particular direction using one or more coils or other pulse emitting components. The frequency of the emitted pulse(s) could be the Larmor frequency of the atomic nuclei in whatever background magnetic field is present. In some examples, a magnetometer (e.g., the magnetometer configured to detect time-varying magnetic fields produced by rotated magnetic spins of the atomic nuclei) could be operated to detect the magnitude of the background magnetic field such that the Larmor frequency could be determined. The emitted pulses could include one or more pi pulses, pi/2 pulses, or other pulses of a magnetic and/or electromagnetic field to rotate or otherwise excite the polarized magnetic moments of the atomic nuclei such that the magnetic spins precess in a manner that can be detected by the magnetometer. For example, the emitted pulse(s) could result in the magnetic spins emitting a free induction decay pulse that decays at a rate related to T2 or other magnetic resonance time constant of the atomic spins and that has a frequency related to the magnitude of the background magnetic field and any inhomogeneities thereof. An orientation of the emitted pulse (i.e., an orientation of the spin-rotating time-varying magnetic field) could be controlled (e.g., by controlling a relating amplitude and/or phase of current pulses applied to respective different pulse-emitting coils) according to a detected direction of the background magnetic field (e.g., such that the magnetic spins of the atomic nuclei are rotated approximately 90 degrees with respect to the direction of the background magnetic field).

The magnetometer could detect the direction, magnitude, magnitude in a particular direction, or other properties of the magnetic field emitted by the rotated magnetic spins of the atomic nuclei. For example, the magnetometer could be a spin-exchange relaxation-free (SERF) magnetometer configured to detect low-frequency components of a magnetic field in one or more specified directions. Additionally or alternatively, the magnetometer could include a multipass scalar atomic magnetometer configured to detect the magnitude of the magnetic field. Additionally or alternatively, the magnetometer could be tuned to detect specific components of the magnetic field (e.g., components within specified range(s) of frequencies) produced by the precessing magnetic spins. For example, the magnetometer could include a radio frequency atomic magnetometer tuned to the Larmor frequency of the magnetic spins of the atomic nuclei. In a particular example, wherein the background magnetic field is the Earth's magnetic field (e.g., between approximately 0.25 and approximately 0.65 Gauss at the Earth's surface), the radio frequency atomic magnetometer could be tuned to a corresponding Larmor frequency of several kilohertz (e.g., approximately 2 kilohertz). Additionally or alternatively, a multipass scalar atomic magnetometer could be configured to detect time-varying magnetic fields at such frequencies.

In some examples, multiple magnetometers could be operated to detect magnetic fields produced by and/or related to magnetized nanoparticles proximate the multiple magnetometers to provide applications described herein. Such multiple magnetometers could be configured and/or operated to detect a magnetic field gradient, to map a magnetic field across an area and/or volume, to determine a magnetic field produced by magnetized nanoparticles in an environment by detecting a magnetic field using a first magnetometer and subtracting a background magnetic field detected by a second magnetometer, or according to some other scheme to provide some other application(s).

Such multiple magnetometers could be configured to detect the same property of magnetic fields at respective locations (e.g., field magnitude, field magnitude in a specified direction, field direction) or different properties. The magnetometers could be similarly configured and/or the same type of magnetometer (e.g., the magnetometers could both be SERF magnetometers, inductive pickup coils, SQUIDS, multipass scalar atomic magnetometers, radio frequency atomic magnetometers) or differently configured. For example, a first magnetometer could be less sensitive than a second magnetometer and the output of the first magnetometer could be used to operate the second magnetometer (e.g., to set a bias, to set an offset, to apply a biasing magnetic field, or to otherwise improve the sensitivity or some other aspect of the operation of the second magnetometer based on information about the magnetic field expected to be detected by the second magnetometer determined from magnetic field information detected by the first magnetometer).

In some examples, a detected and/or determined background field at a particular location could be reduced to improve the operation of a magnetometer to detect a magnetic field of interest (e.g., a magnetic field produced by magnetized nanoparticles proximate the location) at the particular location. This could be performed to reduce a dynamic range required to detect a magnetic field of interest, because a magnetometer is configured to operate in low-field conditions (e.g., the magnetometer is a SERF magnetometer configured to operate in magnetic fields less than some maximum value), or according to some other consideration. In some examples, this could include disposing magnetic shielding and/or shimming materials or components (e.g., components composed of mu-metal, ferrites, conductors, or other magnetic materials) to reduce the effect and/or presence of the background magnetic field at the particular location. In some examples, a biasing magnetic field could be applied to the particular location to cancel the background field. This could include magnets and/or electromagnets configured to provide the cancelling field. In some examples, the cancelling field could be controlled to match the background magnetic field, e.g., by controlling a location and/or orientation of a magnet and/or magnetic material (e.g., shim), by controlling a current applied to an electromagnetic coil, or by some other means.

### IV. EXAMPLE SEPARATION OF NANOPARTICLES

Systems, devices, and methods described herein for detecting properties of magnetized nanoparticles and/or properties of analytes bound thereto can include such nanoparticles having magnetic relaxation times within specified ranges of relaxation times. For example, a system could include a magnetic flux source configured to magnetize such nanoparticles at a first location and a magnetometer to detect magnetic fields related to the magnetized nanoparticles at a second location that is downstream relative to a fluid flow (e.g., a blood flow) that transports the magnetized nanoparticles from the magnetic flux source to the magnetometer. A distance between such a magnetometer and magnetic flux source, a sensitivity of the magnetometer, a strength of the field produced by the magnetic flux source, or some other properties of the system could be specified based on the range of magnetic relaxation times of the nanoparticles, among other factors (e.g., a flow velocity of blood in a portion of subsurface vasculature). Additionally or alternatively, the nanoparticles could include multiple subsets of nanoparticles having magnetic relaxation times within respective ranges of relaxation times to provide some functionality according to an application (e.g., simultaneous detection of multiple respective different analytes).

In such examples, a supply of nanoparticles having relaxation times within such a specified range of relaxation times could be produced according to the specified range of relaxation times. For example, such nanoparticles could be fabricated using self-assembly to have substantially uniform size, geometry, composition, or other properties such that the magnetic relaxation times of the formed nanoparticles are within the specified range of relaxation times. Additionally or alternatively, a source of nanoparticles having magnetic relaxation times spanning a range that exceeds in one or both directions the specified range of relaxation times could be sieved, filtered, or otherwise separated to produce a subset of the provided nanoparticles that have magnetic relaxation times within the specified range of relaxation times.

In some examples, the magnetic relaxation time of a nanoparticle could be related to a size of the particle. For example, a nanoparticle could include a particle of superparamagnetic iron oxide and the magnetic relaxation time of the nanoparticle could be related to the Neel relaxation time of the particle of superparamagnetic iron oxide of the nanoparticles. The Neel relaxation time of a particle of superparamagnetic material can be related to the size of the particle of superparamagnetic material. Thus, the magnetic relaxation time of such nanoparticles could be controlled by controlling a size of the nanoparticles, e.g., by using one or more filters or other size-dependent methods to separate nanoparticles having sizes within a specified range of sizes related to the specified range of magnetic relaxation times. For example, superparamagnetic material particle sizes between approximately 10 nanometers and approximately 20 nanometers could correspond to magnetic relaxation times between approximately 1 second and approximately 1 nanosecond.

Additionally or alternatively, magnetic fields could be applied to a plurality of nanoparticles to separate nanoparticles of the plurality that have magnetic relaxation times within a specified range of relaxation times. In such examples, the nanoparticles could be separated based directly on the magnetic relaxation time of the nanoparticles. That is, the nanoparticles could be magnetized and then a separating magnetic force could be applied during a specified period of time relative to the timing of the magnetization such that the nanoparticles are separated according to their magnetic relaxation times. In some examples, the nanoparticles could be disposed within a region of flow (e.g., a tube or other vessel containing a flowing carrier fluid in which the nanoparticles are disposed) and a first magnetic flux source disposed at a first location could be configured to magnetize the nanoparticles in the region of flow proximate the first location. A second magnetic flux source could be disposed at a second location and configured to apply a separating magnetic force to nanoparticles that were magnetized by the magnetic flux source at the first location, that traveled to the second location, and that are still magnetized when they arrive at the second location. As a result, the separating magnetic force is applied to nanoparticles having relaxation times greater than a specified relaxation time, where the specified relaxation time is related to a distance between the first and second locations and a flow velocity of the carrier fluid within the region of flow.

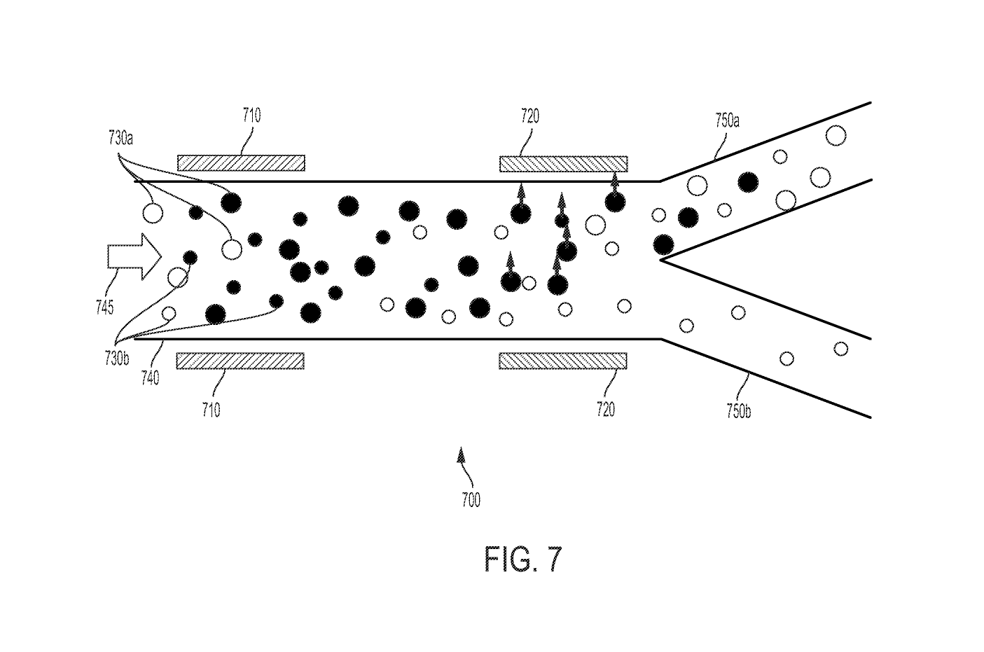

To illustrate such a method of nanoparticle separation, FIG. 7 illustrates an example separation system 700. Example nanoparticles 730a, 703b are disposed in a region of flow 740 (i.e., a cylinder of the system 700 configured to carry a carrier fluid within which the nanoparticles 730a, 703b are disposed). The nanoparticles 730a, 703b include first 730a and second 730b sets of nanoparticles having magnetic relaxation times within respective different ranges of relaxation times. The first nanoparticles 730a have magnetic relaxation times that are longer than the magnetic relaxation times of the second nanoparticles 730b. The region of flow 740 contains a carrier fluid that is flowing (direction of flow indicated by the arrow 745). The carrier fluid could be an aqueous solution, a solution configured to mimic the properties of blood (e.g., a phosphate-buffered saline solution), blood stabilized by an anti-coagulating agent, or some other carrier fluid according to an application. The region of flow 740 is separated into first 750a and second 750b output regions of flow.

The system 700 includes a first magnetic flux source 710 disposed at a first location relative to the region of flow 740 and a second magnetic flux source 720 disposed at a second location relative to the region of flow 740, where the second location is downstream, relative to a direction of flow 745 within the region of flow 740. The first magnetic flux source 710 is configured to configured to produce a magnetic flux and/or field sufficient to at least partially magnetize and/or align a magnetic dipole of proximate nanoparticles 730a, 730b in the region of flow 740 (e.g., by providing a high magnitude magnetic field region of flow 740). The second magnetic flux source 720 is configured to provide a separating magnetic force to proximate magnetized nanoparticles 730a, 730b in the region of flow 740 (e.g., by providing a magnetic field in the region of flow 740 that has a high gradient magnitude).

The fill color in the illustrated nanoparticles 730a, 730b indicates the degree of magnetization of each nanoparticle; a black-filled complex is magnetized while a white-filled complex is substantially not magnetized. Note that magnetization of the nanoparticles 730a, 730b is illustrated at a discrete, binary state for illustration purposes only; the magnetization of the complexes and/or nanoparticles thereof could be continuous-valued or discrete and having a number of possible values greater than 2. Further, a plurality of nanoparticles to be separated could include a population of nanoparticles having magnetic relaxation times across a continuous range of relaxation times. The two sets of nanoparticles 730a, 730b shown in FIG. 7, having magnetic relaxation times within respective ranges of relaxation times, are provided as non-limiting illustrative examples of methods and systems for separating such nanoparticles according to magnetic relaxation time.

As shown in FIG. 7, the nanoparticles 730a, 730b are moved by carrier fluid flow 745 past the first magnetic flux source 710. This can result in the nanoparticle 730a, 730b becoming and/or being magnetized (illustrated by the nanoparticles being more likely to be black-filled, i.e., magnetized, as they pass over the first magnetic flux source 710). Over time, the nanoparticles 730a, 730b become less magnetic (e.g., become non-magnetic, as shown in FIG. 7) according processes related to their magnetic relaxation times. Due to the carrier fluid flow 745, these times are related to distances, within the region of flow 740, from the first magnetic flux source 710. As a result, most of the first nanoparticles 730a remain magnetized at a second location at which the second magnetic flux source 720 is located. Conversely, most of the second nanoparticles 730b are not magnetized at the second location. As a result, substantially only the first nanoparticles 730a experience the separating magnetic force (illustrated by the arrows) exerted by the second magnetic flux source 720.

The separating magnetic force acts to move the first nanoparticles 730a upward, such that substantially only carrier fluid in the first output region of flow 750a contains the first nanoparticles 730a. Further, the second output region of flow contains substantially only the second nanoparticles 730b. Thus, carrier fluid from the first output region of flow 750a could be used as a source of nanoparticles that is enriched in nanoparticles having magnetic relaxation times above a specified value, where the specified value is related to the distance between the first and second locations of respective first 710 and second 720 magnetic flux sources and the flow velocity of carrier fluid in the region of flow 740. Further, carrier fluid from the second output region of flow 750b could be used as a source of nanoparticles that is enriched in nanoparticles having magnetic relaxation times below such a specified value.

Note that the production, by the second magnetic flux source 720, of a separating magnetic force that is directed in a single direction relative the carrier fluid flow 745 (i.e., upward) is intended as a non-limiting example. The second magnetic flux source 720 could apply a separating magnetic force in a different direction or in more than one direction. Related to this, the illustration of the region of flow 740 separating into oppositely-angled output regions of flow 750a, 750b is intended as a non-limiting example, and is related to whatever separating force is applied by the second magnetic flux source 720. For example, the second magnetic flux source 720 could be configured to apply a separating magnetic force from the center of the region of flow 740 toward the walls of the region of flow 740 (e.g., a cylindrically symmetric magnetic separating force). In such examples, the output regions of flow could include concentric pipes, the inner pipe carrying carrier fluid that is enriched nanoparticles that were not substantially affected by the separating magnetic force (e.g., nanoparticles having magnetic relaxation times below some specified value) and the outer output pipe carrying carrier fluid that is enriched nanoparticles that were substantially affected by the separating magnetic force (e.g., nanoparticles having magnetic relaxation times above the specified value).

Further, the second magnetic flux source 720 could be configured to collect proximate magnetized nanoparticles. In such examples, the system 700 could be operated in a non-continuous manner. For example, during a first period of time, carrier fluid containing a plurality of nanoparticles having a range of magnetic relaxation times could be passed through the region of flow 740 and the second magnetic flux source 720 could act to collect, against the walls of the region of flow 740, nanoparticles having magnetic relaxation times greater than some specified value. During a second period of time, carrier fluid containing no nanoparticles could be passed through the region of flow 740 and the second magnetic flux source 720 could be operated to release the collected nanoparticles such that carrier fluid output from the system 700 during the second period of time contains substantially only nanoparticles having magnetic relaxation times greater than the specified value.

A set of nanoparticles having magnetic relaxation times within a specified range of relaxation times could be generated using the methods described herein (e.g., using the system 700) in a variety of ways. In some examples, a first set of nanoparticles having magnetic relaxation times less than a maximum relaxation time of the range of relaxation times could be separated from a source of nanoparticles. A second set of nanoparticles having magnetic relaxation times greater than a minimum relaxation time of the range of relaxation times could then be separated from the first set of nanoparticles. In some examples, such separation could be implemented in a single system, e.g., a system including multiple magnetic flux sources configured to magnetize nanoparticles and multiple magnetic flux sources configured to exert a magnetic force to collect or otherwise separate such magnetized nanoparticles according to magnetic relaxation time.

Further, systems and methods described herein to separate nanoparticles according to magnetic relaxation time could be used multiple times on a carrier fluid containing such nanoparticles to improve a degree and/or specificity of the separation or according to some other application. For example, the carrier fluid of the first output region of flow 750a could be applied to the system 700 one or more further times to reduce the amount of the second nanoparticles 730b in the carrier fluid relative to the amount of the first nanoparticles 730a. This could include connecting the first output region of flow 750a to the input of the region of flow 740 in a loop (e.g., via a pump) such that the separation process is continuous.

Note that, while the system 700 and methods related thereto described herein reference separating nanoparticles that are able to be magnetized and that could be configured to selectively interact with (e.g., bind to) an analyte of interest, these systems and methods could be additionally or alternatively be used to separate elements used to fabricate such nanoparticles. For example, these methods could be used to separate a population of particles of superparamagnetic iron oxide according to magnetic relaxation time. Further, such separation could provide separation of such components according to size or some other property of the components that is related to magnetic relaxation time. Such separated elements could then be used to construct the nanoparticles (e.g., by self-assembly). Such constructed nanoparticles could also be separated according to magnetic relaxation time using the method.

In some examples, magnetic properties of the nanoparticles 730a, 703b (e.g., magnetic relaxation times) can be related to properties (e.g., a pH, an osmolality, a viscosity, a proton content, a Debye length, a degree of adsorption of proteins and other contents of the carrier fluid to the nanoparticles) of a fluid in which the nanoparticles 730a are disposed 730b. In such examples, the region of flow 740 could include a carrier fluid designed to mimic the relevant properties (e.g., in examples wherein the nanoparticles will be used in blood of a person, the region of flow 740 could include blood or blood products from a blood bank).

Note that, while separation of nanoparticles according to magnetic relaxation time is provided in the context of using such nanoparticles to detect analytes in or other properties of a human body, such separation could be applied to other applications. For example, such methods could be used to separate nanoparticles within a specified narrow range of sizes by using these methods to separate nanoparticles having magnetic relaxation times within a range of relaxation times that corresponds to the range of sizes. This could provide for separation of nanoparticles according to size that is improved in some way relative to using a sieve or filter or otherwise separating the nanoparticles by size. For example, nanoparticles separated according to magnetic relaxation time could be more specifically selected, could be separated according to narrower ranges of sizes, or could be improved in some other way.

### V. EXAMPLE WEARABLE DEVICES

Wearable devices as described herein can be configured to be mounted to an external body surface of a wearer and to enable a variety of applications and functions including the detection of magnetic fields produced by magnetized nanoparticles disposed in the body of the wearer (e.g., disposed in a portion of subsurface vasculature of the wearer). Such devices could include one or more magnetic flux sources configured to magnetize such nanoparticles and/or to provide some other functionality (e.g., to polarize the magnetic spins of atomic nuclei in a body). One or more magnetometers of the wearable device could be configured to detect (directly or indirectly) the magnetic fields produced by magnetized nanoparticles disposed proximate the one or more magnetometers (e.g., in portions of subsurface vasculature that are downstream, relative to a direction of blood from, from a location at which the one or more magnetic flux sources magnetize the nanoparticles). Such wearable devices could enable a variety of applications, including measuring properties of the magnetized nanoparticles and/or an analyte with which the nanoparticles are configured to selectively interact (e.g., bind to), to detect other physiological information about a wearer (e.g., heart rate), indicating such measured information or other information to the wearer (e.g., using a vibrator, a screen, a beeper), or other functions.

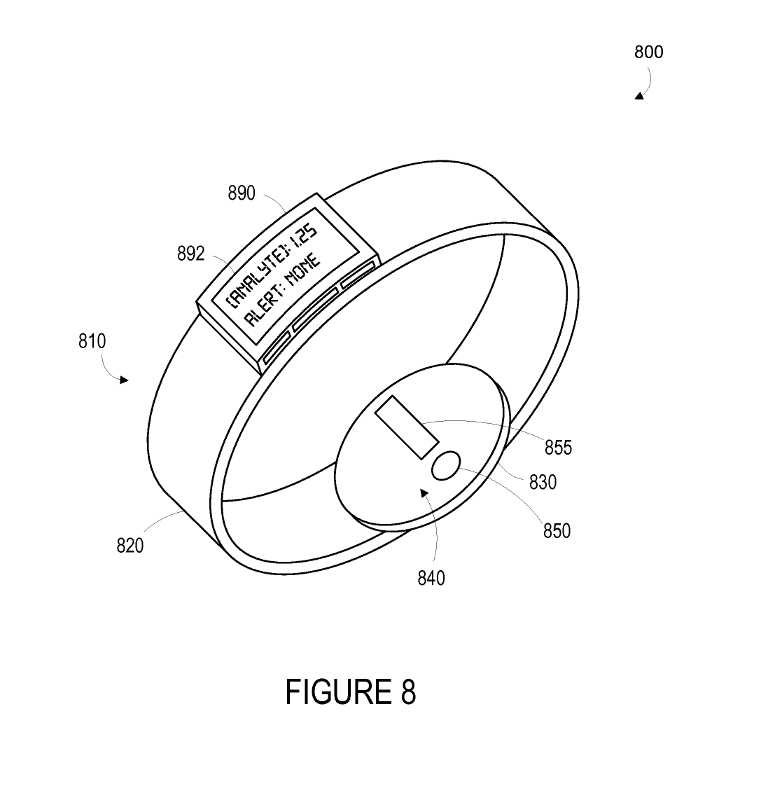

A wearable device 800 (illustrated in FIG. 8) can be configured to magnetize nanoparticles disposed in a wearer's body (e.g., disposed in portions of subsurface vasculature proximate the device 800) and to detect magnetic fields produced by such magnetized nanoparticles disposed in the wearer's body or other physiological parameters of a person wearing the device. The term “wearable device,” as used in this disclosure, refers to any device that is capable of being worn at, on or in proximity to a body surface, such as a wrist, ankle, waist, chest, or other body part. In order to take in vivo measurements in a non-invasive manner from outside of the body, the wearable device may be positioned on a portion of the body where subsurface vasculature or other targets or elements of the body of the wearer are easily observable, the qualification of which will depend on the type of detection system used. The device may be placed in close proximity to the skin or tissue. A mount 810, such as a belt, wristband, ankle band, etc. can be provided to mount the device at, on or in proximity to the body surface. The mount 810 may prevent the wearable device from moving relative to the body to reduce measurement error and noise. In one example, shown in FIG. 8, the mount 810, may take the form of a strap or band 820 that can be worn around a part of the body. Further, the mount 810 may be an adhesive substrate for adhering the wearable device 800 to the body of a wearer.

A housing 830 is disposed on the mount 810 such that it can be positioned on the body. A contact surface 840 of the housing 830 is intended to be mounted facing to the external body surface. The housing 830 may include a magnetic flux source 855 for producing a magnetic field sufficient to magnetize nanoparticles disposed in the body of the wearer (e.g., magnetized nanoparticles disposed in portions of subsurface vasculature). The housing 830 may additionally include a magnetometer 850 for detecting magnetic fields produced by such magnetized nanoparticles disposed in the body of the wearer. The housing 830 could be configured to be water-resistant and/or water-proof. That is, the housing 830 could be configured to include sealants, adhesives, gaskets, welds, transparent windows, apertures, press-fitted seams, and/or other joints such that the housing 830 was resistant to water entering an internal volume or volumes of the housing 830 when the housing 830 is exposed to water. The housing 830 could further be water-proof, i.e., resistant to water entering an internal volume or volumes of the housing 830 when the housing 830 is submerged in water. For example, the housing 830 could be water-proof to a depth of 1 meter, i.e., configured to resist water entering an internal volume or volumes of the housing 830 when the housing 830 is submerged to a depth of 1 meter.

The magnetic flux source 855 is configured to produce a magnetic field sufficient to magnetize nanoparticles disposed proximate to the magnetic flux source 855 in an environment of interest, e.g., a portion of subsurface vasculature of a wearer. For example, the magnetic flux source 855 could be configured to produce a magnetic field having a magnitude of several hundred Gauss (e.g., greater than approximately 100 Gauss) at a distance of approximately 1 centimeter from the contact surface 840 (e.g., a distance within which a portion of subsurface vasculature containing the nanoparticles may be located when the device 800 is mounted to a body). The magnitude of the magnetic field produced by the magnetic flux source 855 and the dimensions of the magnetic flux source 855 (e.g., the length of the magnetic flux source 855 in a direction aligned with a direction of the portion of subsurface vasculature) could be specified such that nanoparticles flowing in the body proximate to the magnetic flux source 855 are magnetized. In an illustrative example, the nanoparticles could have a magnetic relaxation time of approximately 1 second and could be disposed in a blood flow having a flow velocity of several centimeters per second. In such an example, a magnetic flux source as described herein could have a length, in the direction of the blood flow, of several centimeters such that the nanoparticles are maintained proximate to the magnetic flux source for a sufficient period of time to be magnetized by the magnetic flux source.

Note that the magnetic flux source 855 could be configured to provide some other functionality, e.g., to polarize the magnetic spins of atomic nuclei such that the magnetic field in the environment of such atomic nuclei (e.g., a magnetic field produced by a magnetized nanoparticle proximate such atomic nuclei) could be detected (e.g., by the magnetometer 850 detecting time-varying magnetic and/or electromagnetic fields produced by such atomic nuclei through nuclear magnetic resonance). In another example, the magnetic flux source 855 could be configured to collect magnetized nanoparticles and/or to release such collected magnetized nanoparticles, e.g., to facilitate extraction of the collected nanoparticles from the body, to provide a higher-magnitude signal for the magnetometer 850 to detect, or according to some other application. The magnetic flux source 855 could include one or more electromagnets, permanent magnets, or other magnetic producing elements. Further, the magnetic flux source 855 could be configured and/or operated to change a magnetic field produced by the magnetic flux source 855, e.g., to reduce a magnitude of a produced magnetic field that is detected by the magnetometer 850, to reduce an inhomogeneity of the magnetic field proximate the magnetometer 850 that is caused by the magnetic flux source 855, or according to some other application. This could include changing a current applied to an electromagnet of the magnetic flux source 855, mechanically actuating an electromagnet, permanent magnet, or other flux producing element of the magnetic flux source 855, or performing some other operation(s).

The magnetometer 850 is configured to detect a magnetic field produced by magnetized nanoparticles, precessing magnetic spins of atomic nuclei, or other magnetic-field-producing elements disposed proximate the magnetometer (e.g., within from approximately 1 millimeter to approximately 1 centimeter) in an environment of interest, e.g., a portion of subsurface vasculature of a wearer. The magnetometer 850 could be configured to have a sensitivity such that the magnetometer can detect changes in a measured magnetic field of less than approximately 10 femtoteslas. The magnetometer could be configured to detect a direction, magnitude, property of change over time, or some other property of the magnetic fields produced by the magnetized nanoparticles. The magnetometer 850 could be configured to detect time-varying magnetic fields across a specified range of frequencies, e.g., less than several kilohertz (e.g., a spin-exchange relaxation-free atomic magnetometer, a multi-pass scalar atomic magnetometer), at a particular frequency (e.g., a radio-frequency atomic magnetometer tuned to a frequency of interest, e.g., an expected frequency of precession of magnetic spins of atomic nuclei in a magnetic field).

The wearable device 800 could include one or more bias coils, magnets, shims, magnetic shielding elements, or other components to reduce a background magnetic field to which the magnetometer 850 is exposed (e.g., to cancel the effects of the Earth's magnetic field on the magnetometer 850, to cancel the effects of the magnetic flux source 855 on the magnetometer), to reduce an inhomogeneity of the magnetic field in an environment of interest (e.g., to reduce an inhomogeneity in the earth's magnetic field in a portion of subsurface vasculature proximate the magnetometer 850), and/or to provide some other functionality. Additionally or alternatively, in examples wherein a magnetic field produced by the magnetic flux source 855 interferes with the operation of the magnetometer 850 to detect properties of the magnetized nanoparticles and/or an analyte bound thereto (e.g., wherein the magnetic flux source 855 creates an inhomogeneity in the Earth's magnetic field proximate the magnetometer 850, wherein the flux source 855 creates a magnetic field at the location of the magnetometer 850 that interferes with measurement of a magnetic field produced by and/or affected by the magnetized nanoparticles), the magnetic flux source 855 could be intermittently operated to produce such a magnetic field (e.g., a current applied to an electromagnet of the magnetic flux source 855 could be reduced or zeros during certain periods of time wherein the magnetometer 850 could operate to detect magnetic fields).

The magnetometer 850 could be configured to detect an oscillating or otherwise time-varying magnetic field produced by the magnetized nanoparticles in response to exposure to an oscillating magnetic field produced by an excitation coil or other component (e.g., antenna) of the wearable device 800. In some examples, this could include the magnetometer including one or more inductive pickup coils configured to detect the produced oscillating or otherwise time-varying magnetic fields and/or to emit the oscillating magnetic field produced by the wearable device 800 (i.e., the excitation coil used to produce the oscillating magnetic field in the environment of interest is also part of the magnetometer and used to detect the oscillating or otherwise time-varying magnetic fields responsively produced by the magnetized nanoparticles). Additionally or alternatively, such a responsively produced magnetic field could be detected by an optical atomic magnetometer (e.g., a SERF, a multipass scalar atomic magnetometer, a radio-frequency atomic magnetometer). For example, a magnitude of a responsively produced time-varying magnetic field could be detected by a radio-frequency atomic magnetometer tuned to the frequency of the responsively produced time-varying magnetic field, e.g., to a frequency of a harmonic of the oscillating magnetic field produced by the device 800.

The magnetometer 850 could be configured to detect an oscillating or otherwise time-varying magnetic and/or electromagnetic field produced by magnetic spins of atomic nuclei that are precessing proximate the magnetometer 850. That is, the magnetometer 850 could be configured to detect, using the techniques of nuclear magnetic resonance, the magnetic field strength in the environment of the atomic nuclei, e.g., of hydrogen atoms in water or other chemicals in a portion of subsurface vasculature. For example, the magnetometer 850 could include one or more pulse emitters (e.g., electromagnetic coils) configured to emit time-varying magnetic fields (e.g., pi pulses, pi/2 pulses, other waveforms used in nuclear magnetic resonance and/or magnetic resonance imaging) to rotate the magnetic spins of the atomic nuclei. The background magnetic field proximate the magnetometer 850 could be substantially homogeneous (e.g., could be the Earth's magnetic field) such that the rotated magnetic spins of the atomic nuclei precess at approximately the same frequency (e.g., at a frequency related to strength of the magnetic field proximate each of the atomic nuclei). The magnetic field produced by a magnetized nanoparticle could alter the precession frequency of atomic nuclei proximate the magnetized nanoparticle. The magnetometer could detect a time-varying magnetic field produced by the precessing magnetic spins of the atomic nuclei and such detected information could be used to determine a property of the magnetized nanoparticles and/or of an analyte bound thereto.

The magnetometer could include a variety of components configured in a variety of ways to detect one or more properties of a magnetic field produced by and/or related to magnetized nanoparticles. The magnetometer could include a superconducting quantum interference device (SQUID), spin-exchange relaxation-free (SERF) magnetometer, a multipass scalar atomic magnetometer, a radio-frequency atomic magnetometer, one or more inductive loops or coils or other antenna structures, a spin precession magnetometer, or some other magnetic-field-detecting components or devices. In examples wherein the magnetometer 850 includes elements having a very high temperature (e.g., an alkali vapor cell of a SERF, multipass scalar atomic magnetometer, and/or radio-frequency atomic magnetometer) or a very low temperature (e.g., the Josephson junction(s) of a SQUID), the magnetometer 850 and/or the housing 810 could include means for insulating the high- or low-temperature elements or for otherwise controlling the temperature of such elements and/or preventing injury to a user due to exposure to extreme temperatures of such elements. For example, an alkali vapor cell and/or other laments of a SERF magnetometer could be wholly or partially contained in an evacuated volume (e.g., a dewar), insulated with an aerogel, or otherwise insulated.

The wearable device 800 may also include a user interface 890 via which the wearer of the device may receive one or more recommendations or alerts generated either from a remote server or other remote computing device, or from a processor within the device. The alerts could be any indication that can be noticed by the person wearing the wearable device. For example, the alert could include a visual component (e.g., textual or graphical information on a display), an auditory component (e.g., an alarm sound), and/or tactile component (e.g., a vibration). Further, the user interface 890 may include a display 892 where a visual indication of the alert or recommendation may be displayed. The display 892 may further be configured to provide an indication of the measured magnetic field and/or one or more determined properties of the magnetized nanoparticles and/or an analyte in the body of the wearer.

Note that example devices herein are configured to be mounted to a wrist of a wearer. However, the embodiments described herein could be applied to other body parts (e.g., an ankle, a thigh, a chest, a forehead, a thigh, a finger), or to detect magnetic fields produced by magnetized nanoparticles in other environments. For example, embodiments described herein could be applied to detect one or more properties in a target environment (e.g., a natural environment, an environment of an industrial, pharmaceutical, or water treatment process).

Wearable devices and other embodiments as described herein can include a variety of components configured in a variety of ways. Devices described herein could include electronics including a variety of different components configured in a variety of ways to enable applications of the wearable device. The electronics could include controllers, amplifiers, switches, display drivers, touch sensors, wireless communications chipsets (e.g., Bluetooth radios or other radio transceivers and associated baseband circuitry to enable wireless communications between the wearable device and some other system(s)), or other components. The electronics could include a controller configured to operate one or more magnetic flux sources, magnetometers and/or other sensors to detect a magnetic field and/or to detect some other properties of a wearer or to perform some other functions. The controller could include a processor configured to execute computer-readable instructions (e.g., program instructions stored in data storage of the wearable device) to enable applications of the wearable device. The electronics can include additional or alternative components according to an application of the wearable device.

Wearable devices as described herein could include one or more user interfaces. A user interface could include a display configured to present an image to a wearer and to detect one or more finger presses of a wearer on the interface. The controller or some other component(s) of the electronics could operate the user interface to provide information to a wearer or other user of the device and to enable the wearer or other user to affect the operation of the wearable device, to determine some property of the wearable device and/or of the wearer of the wearable device (e.g., a concentration of an analyte in the blood of the wearer determined based on a detected magnetic field and/or a health state of a wearer of the wearable device), or to provide some other functionality or application to the wearer and/or user. As one example, the wearer could press an indicated region of the user interface to indicate that the wearable device should begin logging detected medical information about the wearer. Other indicated information, changes in operation of the wearable device, or other functions and applications of the user interface are anticipated.

Note that the embodiments illustrated in the Figures are illustrative examples and not meant to be limiting. Alternative embodiments, including more or fewer components in alternative configurations are anticipated. A wearable device could include multiple housings or other such assemblies each containing some set of components to enable applications of such a wearable device. For example, a wearable device could include a first housing within which are disposed one or more magnetic flux sources configured to magnetize nanoparticles disposed in the wearer's body (e.g., within portions of subsurface vasculature of the wearer) and one or more magnetometers configured to detect magnetic fields produced such magnetized nanoparticles. The wearable device could additionally include a second housing containing a user interface and electronics configured to operate the magnetic flux source(s) and magnetometer(s) and to present information to and receive commands from a user of the wearable device. A wearable device could be configured to perform a variety of functions and to enable a variety of applications. Wearable devices could be configured to operate in concert with other devices or systems; for example, wearable devices could include a wireless communication interface configured to transmit data indicative of one or more properties of the body of a wearer of the wearable device. Other embodiments, operations, configurations, and applications of a wearable device as described herein are anticipated.

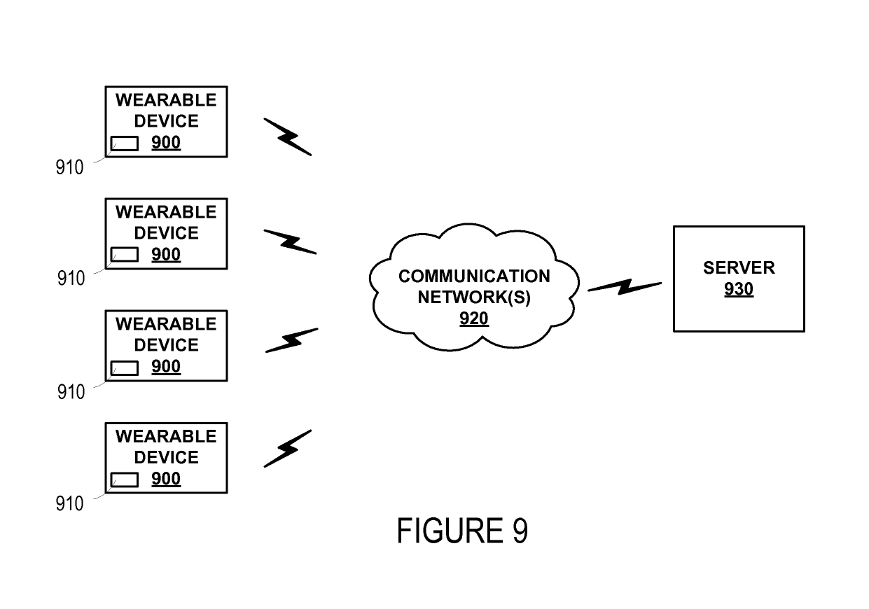

FIG. 9 is a simplified schematic of a system including one or more wearable devices 900. The one or more wearable devices 900 may be configured to transmit data via a communication interface 910 over one or more communication networks 920 to a remote server 930. In one embodiment, the communication interface 910 includes a wireless transceiver for sending and receiving communications to and from the server 930. In further embodiments, the communication interface 910 may include any means for the transfer of data, including both wired and wireless communications. For example, the communication interface may include a universal serial bus (USB) interface or a secure digital (SD) card interface. Communication networks 620 may be any one of may be one of: a plain old telephone service (POTS) network, a cellular network, a fiber network and a data network. The server 930 may include any type of remote computing device or remote cloud computing network. Further, communication network 920 may include one or more intermediaries, including, for example wherein the wearable device 900 transmits data to a mobile phone or other personal computing device, which in turn transmits the data to the server 930.

In addition to receiving communications from the wearable device 900, such as detected magnetic fields produced by magnetized nanoparticles disposed in a body of a wearer (e.g., disposed in portion(s) of subsurface vasculature of a wearer) and/or information determined therefrom (e.g., information about an analyte with which the nanoparticles are configured to selectively interact) or other collected physiological properties and data, the server may also be configured to gather and/or receive either from the wearable device 900 or from some other source, information regarding a wearer's overall medical history, environmental factors and geographical data. For example, a user account may be established on the server for every wearer that contains the wearer's medical history. Moreover, in some examples, the server 930 may be configured to regularly receive information from sources of environmental data, such as viral illness or food poisoning outbreak data from the Centers for Disease Control (CDC) and weather, pollution and allergen data from the National Weather Service. Further, the server may be configured to receive data regarding a wearer's health state from a hospital or physician. Such information may be used in the server's decision-making process, such as recognizing correlations and in generating clinical protocols.

Additionally, the server may be configured to gather and/or receive the date, time of day and geographical location of each wearer of the device during each measurement period. Such information may be used to detect and monitor spatial and temporal spreading of diseases. As such, the wearable device may be configured to determine and/or provide an indication of its own location. For example, a wearable device may include a GPS system so that it can include GPS location information (e.g., GPS coordinates) in a communication to the server. As another example, a wearable device may use a technique that involves triangulation (e.g., between base stations in a cellular network) to determine its location. Other location-determination techniques are also possible.

The server may also be configured to make determinations regarding the efficacy of a drug or other treatment based on information regarding the drugs or other treatments received by a wearer of the device and, at least in part, the detected magnetic field data and the indicated health state of the user. From this information, the server may be configured to derive an indication of the effectiveness of the drug or treatment. For example, if a drug is intended to treat nausea and the wearer of the device does not indicate that they are experiencing nausea after beginning a course of treatment with the drug, the server may be configured to derive an indication that the drug is effective for that wearer. In another example, a wearable device may be configured to detect cancer cells by detecting properties of magnetized nanoparticles that are configured to selectively interact with cancer cells. If a wearer is prescribed a drug intended to destroy cancer cells, but the server receives data from the wearable device indicating that the number of cancer cells in the wearer's blood has been increasing over a certain number of measurement periods, the server may be configured to derive an indication that the drug is not effective for its intended purpose for this wearer.

Further, some embodiments of the system may include privacy controls which may be automatically implemented or controlled by the wearer of the device. For example, where a wearer's collected magnetic field data and health state data are uploaded to a cloud computing network for trend analysis by a clinician, the data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined.

Additionally or alternatively, wearers of a device may be provided with an opportunity to control whether or how the device collects information about the wearer (e.g., information about a user's medical history, social actions or activities, profession, a user's preferences, or a user's current location), or to control how such information may be used. Thus, the wearer may have control over how information is collected about him or her and used by a clinician or physician or other user of the data. For example, a wearer may elect that data, such as health state and detected magnetic field data, collected from his or her device may only be used for generating an individual baseline and recommendations in response to collection and comparison of his or her own data and may not be used in generating a population baseline or for use in population correlation studies.

### VI. EXAMPLE ELECTRONICS PLATFORM FOR A DEVICE

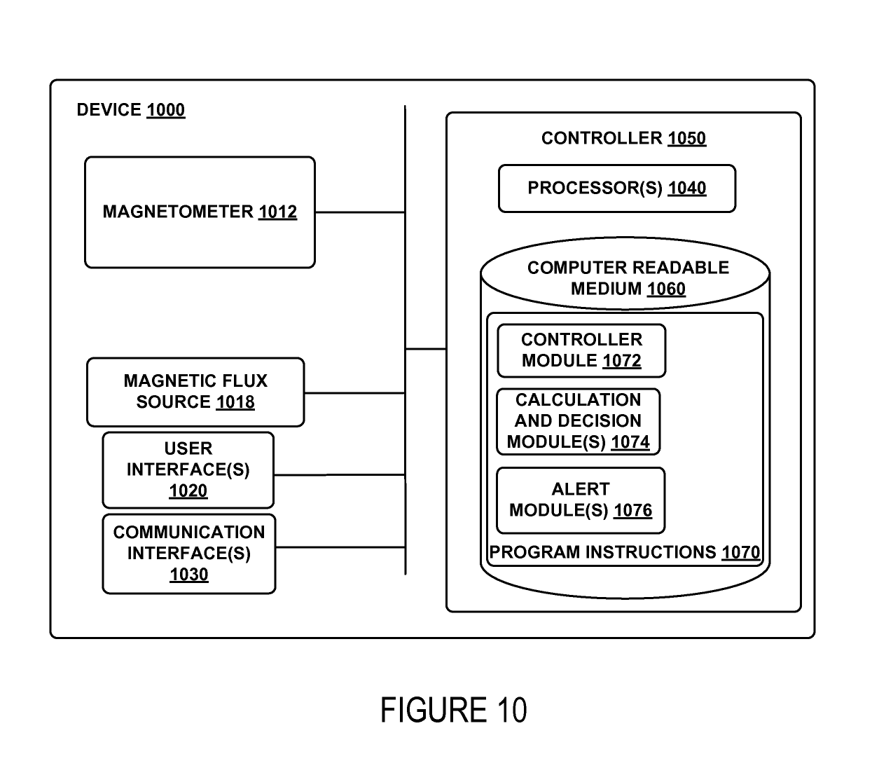

FIG. 10 is a simplified block diagram illustrating the components of a device 1000, according to an example embodiment. Device 1000 may take the form of or be similar to one of the wearable devices 100, 200, 300, 400, 500, or 800 shown in FIGS. 1, 2, 3, 4, 5A-B, and 8. However, device 1000 may also take other forms, such as an ankle, waist, or chest-mounted device. Device 1000 could also take the form of a device that is not configured to be mounted to a body. For example, device 1000 could take the form of a handheld device configured to be maintained in proximity to an environment of interest (e.g., a body part, a biological sample container, a volume of a water treatment system) by a user or operator of the device 1000 or by a frame or other supporting structure. In some examples, device 1000 could be or could form part of device configured to detect properties of an ex vivo and/or in vitro environment (e.g., the device 1000 could be configured to be operated as part of a flow cytometry experiment). Device 1000 also could take other forms.

In particular, FIG. 10 shows an example of a device 1000 having a magnetometer 1012, a magnetic flux source 1018, a user interface 1020, communication interface 1030 for transmitting data to a remote system, and a controller 1050. The components of the device 1000 may be disposed on a mount or on some other structure for mounting the device to enable stable detection of one or more properties (e.g., magnetic fields produced by magnetized nanoparticles) of an environment of interest (e.g., of a body of a wearer of the device 1000), for example, mounting to an external body surface where one or more portions of subsurface vasculature or other anatomical elements are readily observable.

Controller 1050 may be provided as a computing device that includes one or more processors 1040. The one or more processors 1040 can be configured to execute computer-readable program instructions 1070 that are stored in the computer readable data storage 1060 and that are executable to provide the functionality of a device 1000 described herein.

The computer readable medium 1060 may include or take the form of one or more non-transitory, computer-readable storage media that can be read or accessed by at least one processor 1040. The one or more computer-readable storage media can include volatile and/or non-volatile storage components, such as optical, magnetic, organic or other memory or disc storage, which can be integrated in whole or in part with at least one of the one or more processors 1040. In some embodiments, the computer readable medium 1060 can be implemented using a single physical device (e.g., one optical, magnetic, organic or other memory or disc storage unit), while in other embodiments, the computer readable medium 1060 can be implemented using two or more physical devices.

The magnetometer 1012 is configured to detect a magnetic field produced by and/or related to magnetized nanoparticles disposed proximate the magnetometer (e.g., within from approximately 1 millimeter to approximately 1 centimeter) in an environment of interest, e.g., a portion of subsurface vasculature of a wearer. The magnetometer could be configured to have a sensitivity such that the magnetometer can detect changes in a measured magnetic field of less than approximately 10 femtoteslas. The magnetometer could include one or more inductive pickup coils configured to detect an oscillating or otherwise time-varying magnetic field produced by the magnetized nanoparticles in response to exposure to an oscillating magnetic field produced by an excitation coil or some other component (e.g., antenna) of the device 1000. The magnetometer could include more or more pulse emitters (e.g., electromagnetic coils) configured to emit pulses or other patterns of magnetic field into the environment of interest (e.g., pi pulse, pi/2 pulses) to rotate magnetic spins of atomic nuclei in the environment of interest. The magnetometer 1012 could then detect time-varying magnetic and/or electromagnetic fields generated by the rotated magnetic moments of the atomic nuclei as the magnetic moments precess in response to the rotation. A frequency, frequency spectrum, or other properties of the detected time-varying field could be related to the presence, location, orientation, amount, degree of aggregation, or other properties of magnetized nanoparticles in the environment of interest.

The magnetometer could include amplifiers, oscillators, ADCs, switches, filters, light emitter, light detectors, or other components configured to detect a magnetic field using one or more magnetic-field-sensitive elements of the magnetometer 1012. For example, the magnetometer 1012 could be a SERF magnetometer, a multipass scalar atomic magnetometer, a radio-frequency atomic magnetometer, or some other variety of atomic magnetometer that includes an alkali vapor cell (i.e., an enclosed volume containing a high-pressure, high-temperature vapor that includes alkali metal atoms) and the electronics could include a heater configured to vaporize the alkali metal in the vapor cell, a pump laser configured to emit circularly polarized light into the vapor cell to align the alkali metal atoms, a probe laser configured to probe the aligned alkali atoms with linearly polarized light, and a light detector configured to detect the change in orientation of the linearly polarized light that is related to the detected magnetic field. Other examples of magnetometers and electronics thereof are anticipated.

The device 100 could include a bias coil (not shown) that is configured to produce a bias magnetic field to reduce a background magnetic field to which the magnetometer 1012 is exposed and/or to reduce an inhomogeneity of the magnetic field in the environment of interest (e.g., to cancel the effects of the Earth's magnetic field on the magnetometer 1012, to cancel the effects of the magnetic flux source 1018 on the magnetometer 1012) and/or to provide some other functionality. The bias coil could be driven according to a bias field magnitude determined based on an output of the magnetometer 1012, an output of some other magnetometer (not shown), an output of an accelerometer, gyroscope, or some other sensor, or based on some other consideration.

The magnetic flux source 1018 is configured to produce magnetic field sufficient to magnetize nanoparticles proximate the device 1000 (e.g., proximate the magnetic flux source 1018) that are upstream, relative to a direction of flow in the environment of interest (e.g., a direction of a blood flow in a portion of subsurface vasculature), from the location of the magnetometer 1012. Such magnetized nanoparticles can flow downstream to be detected by the magnetometer 1012 (e.g., by detecting a magnetic field produced by and/or affected by the magnetized nanoparticles). The magnetic flux source 1018 could be a permanent magnet and/or an electromagnet. In some examples, the magnetic flux source 1018 could be operated to collect nanoparticles (e.g., by exerting an attractive magnetic force) during a first period of time and subsequently to release the collected nanoparticles (e.g., to allow detection, by the magnetometer 1012, of a magnetic field produced by and/or affected by the collected nanoparticles). In some examples, the magnetic flux source 1018 and/or some other source of magnetic flux could be configured to polarize magnetic spins of atomic nuclei in the environment of interest such that the magnetometer 1012 can detect the presence or other properties of the magnetized nanoparticles by rotating the polarized magnetic spins of the atomic nuclei and detecting a time-varying magnetic field produced by precession of the rotated magnetic spins of the atomic nuclei.

Note that a device could include a subset of the elements described here, e.g., a device could lack a bias coil, excitation coil, magnetic flux source, and/or some other combination of elements. Further, a device could include multiple of one or more illustrated elements. For example, a device could include multiple magnetometers configured to detect a magnetic field at respective multiple different locations and/or in multiple different directions. In another example, a device could include multiple bias coils to cancel magnetic fields in multiple different directions and/or for multiple different magnetometers. In some examples, multiple illustrated elements of the device 1000 could be implemented as the same component and/or share some component(s) in common.

The program instructions 1070 stored on the computer readable medium 1060 may include instructions to perform any of the methods described herein. For instance, in the illustrated embodiment, program instructions 1070 include a controller module 1072, calculation and decision module 1074 and an alert module 1076.

Calculation and decision module 1074 may include instructions for operating the magnetometer 1012 and/or some other components (e.g., one or more bias coils, pulse emitters, and/or excitation coils 1016) to detect magnetic fields produced by and/or affected by magnetized nanoparticles proximate the magnetometer 1012 and analyzing data generated by the magnetometer 1012 to determine information about magnetized nanoparticles and/or analytes in a body (e.g., by detecting pulses or other features in a detected magnetic field, a detected T2* of atomic nuclei, or other detected parameters related to aggregates of magnetized nanoparticles in the change of a detected magnetic field over time) or other information (e.g., health states) of a body of a wearer of the device 1000, such as a concentration of an analyte in blood of the body at a plurality of points in time. Calculation and decision module 1074 can additionally include instructions for analyzing the data to determine if a medical condition or other specified condition is indicated, or other analytical processes relating to the environment proximate to the device 1000. In particular, the calculation and decision module 1074 may include instructions for operating a bias coil to reduce a magnetic field detected by the magnetometer 1012, instructions for operating an excitation coil to produce an oscillating or otherwise time-varying magnetic field in an environment containing magnetized nanoparticles, for operating a pulse emitter to rotate magnetic spins of atomic nuclei, or for performing some other operations. These instructions could be executed at each of a set of preset measurement times.

The controller module 1072 can also include instructions for operating a user interface 1020. For example, controller module 1072 may include instructions for displaying data collected by the data collection system 1010 and analyzed by the calculation and decision module 1074, or for displaying one or more alerts generated by the alert module 1076. Controller module 1072 may include instructions for displaying data related to a detected magnetic field produced by and/or affected by magnetized nanoparticles in one or more portions of subsurface vasculature or some other detected and/or determined health state of a wearer. Further, controller module 1072 may include instructions to execute certain functions based on inputs accepted by the user interface 1020, such as inputs accepted by one or more buttons disposed on the user interface.

Communication interface 1030 may also be operated by instructions within the controller module 1072, such as instructions for sending and/or receiving information via a wireless antenna, which may be disposed on or in the device 1000. The communication interface 1030 can optionally include one or more oscillators, mixers, frequency injectors, etc. to modulate and/or demodulate information on a carrier frequency to be transmitted and/or received by the antenna. In some examples, the device 1000 is configured to indicate an output from the processor by modulating an impedance of the antenna in a manner that is perceivable by a remote server or other remote computing device.

The program instructions of the calculation and decision module 1074 may, in some examples, be stored in a computer-readable medium and executed by a processor located external to the device 1000. For example, the device 1000 could be configured to collect certain data regarding magnetic fields produced by and/or affected by magnetized nanoparticles disposed in the body of the user and then transmit the data to a remote server, which may include a mobile device, a personal computer, the cloud, or any other remote system, for further processing.

The computer readable medium 1060 may further contain other data or information, such as medical and health history of a user of the device 1000, that may be useful in determining whether a medical condition or some other specified condition is indicated. Further, the computer readable medium 1060 may contain data corresponding to certain physiological parameter baselines, above or below which a medical condition is indicated. The baselines may be pre-stored on the computer readable medium 1060, may be transmitted from a remote source, such as a remote server, or may be generated by the calculation and decision module 1074 itself. The calculation and decision module 1074 may include instructions for generating individual baselines for the user of the device 1000 based on data collected over a certain number of measurement periods. Baselines may also be generated by a remote server and transmitted to the device 1000 via communication interface 1030. The calculation and decision module 1074 may also, upon determining that a medical or other emergency condition is indicated, generate one or more recommendations for the user of the device 1000 based, at least in part, on consultation of a clinical protocol. Such recommendations may alternatively be generated by the remote server and transmitted to the device 1000.

In some examples, the collected magnetic field data, baseline profiles, health state information input by device users and generated recommendations and clinical protocols may additionally be input to a cloud network and be made available for download by a user's physician. Trend and other analyses may also be performed on the collected data, such as analyte and/or nanoparticle data and health state information, in the cloud computing network and be made available for download by physicians or clinicians.

Further, detected magnetic field data and determined magnetized nanoparticle, analyte, and health state data from individuals or populations of device users may be used by physicians or clinicians in monitoring efficacy of a drug or other treatment. For example, high-density, real-time data may be collected from a population of device users who are participating in a clinical study to assess the safety and efficacy of a developmental drug or therapy. Such data may also be used on an individual level to assess a particular wearer's response to a drug or therapy. Based on this data, a physician or clinician may be able to tailor a drug treatment to suit an individual's needs.

In response to a determination by the calculation and decision module 1074 that a medical or other specified condition is indicated, the alert module 1076 may generate an alert via the user interface 1020. The alert may include a visual component, such as textual or graphical information displayed on a display, an auditory component (e.g., an alarm sound), and/or tactile component (e.g., a vibration). The textual information may include one or more recommendations, such as a recommendation that the user of the device contact a medical professional, seek immediate medical attention, or administer a medication.

### VII. EXAMPLE METHODS

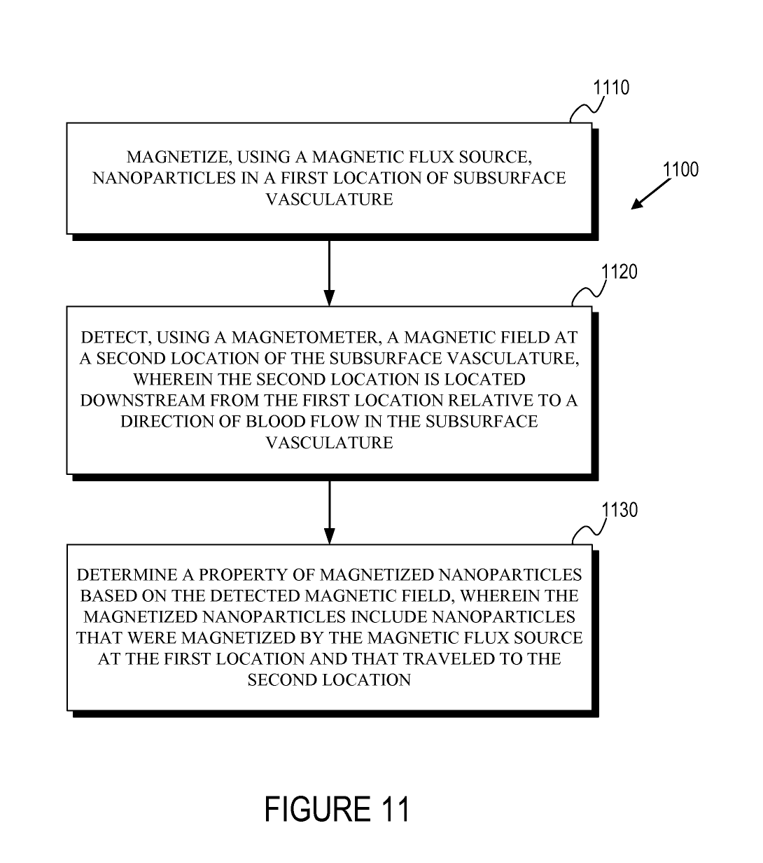

FIG. 11 is a flowchart of an example method 1100 for detecting properties of nanoparticles and/or an analyte bound thereto in a biological environment by detecting a magnetic field produced by and/or affected by the nanoparticles. The method 1100 includes magnetizing, using a magnetic flux source, nanoparticles in a first location of subsurface vasculature (1110). This could include the magnetic flux source producing a magnetic field in the first location of a portion of subsurface vasculature having a sufficient magnetic field magnitude (e.g., greater than approximately 100 Gauss) to at least partially magnetize, to align one or more magnetic moments of (e.g., magnetic moments of one or more nanoparticles of superparamagnetic iron oxide of), to rotate, or to otherwise magnetize the nanoparticles. Magnetizing the nanoparticles (1110) could include applying current to an electromagnet of the magnetic flux source, rotating, translation, or otherwise actuating a permanent magnet, magnetic shim, or other element of the magnetic flux source, moving the magnetic flux source proximate to the portion of subsurface vasculature, or performing some other steps to produce a magnetic field sufficient to magnetize the nanoparticles.

The method 1100 includes detecting, using a magnetometer, a magnetic field at a second location of the subsurface vasculature, wherein the second location is located downstream from the first location relative to a direction of blood flow in the subsurface vasculature (1120). This could include detecting a magnitude, direction, magnitude in a particular direction, a pattern or property of change over time of a property of the magnetic field, or some other property of the produced magnetic field. The detected magnetic field could be directly related to the magnetic field produced by the magnetized nanoparticles (e.g., could be a field generated by a magnetic moment of the magnetized nanoparticles, could be produced by the nanoparticles in response to an applied external energy, e.g., an applied oscillating magnetic field). The detected magnetic field could be directly related to the magnetic field produced by the magnetized nanoparticles (e.g., could be a magnetic field produced by rotated, precessing magnetic spins of atomic nuclei proximate the magnetized nanoparticles, where a frequency, coherence, or other properties of the precession is related to the magnetic field produced by the magnetized nanoparticles). Detecting the magnetic field (1120) could include producing an oscillating magnetic field in the second location of subsurface vasculature and detecting a time-varying magnetic field responsively reflected, phase-shifted, frequency-shifted, frequency-multiplied, or otherwise produced by the magnetized nanoparticles. Detecting the magnetic field (1120) could include applying a bias magnetic field (e.g., by operating a bias coil disposed proximate the magnetometer) to cancel a background magnetic field (e.g., a magnetic field produced by the Earth) to which the magnetometer is exposed. Detecting the magnetic field (1120) could include rotating magnetic spins of atomic nuclei in the second location of subsurface vasculature (e.g., by emitting a magnetic or electromagnetic pulse at the Larmor frequency of the magnetic spins of the atomic nuclei, e.g., a pi pulse, a pi/2 pulse) and detecting magnetic fields produced by the responsively precessing rotated magnetic spins.

The method 1100 additionally includes determining a property of the magnetized nanoparticles based on the detected magnetic field, wherein the magnetized nanoparticles include nanoparticles that were magnetized by the magnetic flux source at the first location and that traveled to the second location (1130). This could include determining the orientation and/or location of one or more of the magnetized nanoparticles, a degree of aggregation of the magnetized nanoparticles, or the detection of some other property of the magnetized nanoparticles. Determining a property of the magnetized nanoparticles (1130) could include determining and/or detecting features of the detected magnetic field, e.g., detecting the amplitude, width, timing, decay rate or decay time constant, frequency spectrum or Fourier transform, or other properties of the detected magnetic field over time. Further, such determined properties of the magnetized nanoparticles could be related to properties of an analytes of interest with which the magnetized nanoparticles are configured to selectively interact (e.g., to bind to). For example, multiple magnetized nanoparticles could bind to a single instance of an analyte (e.g., to a single cancer cell) such that detection of an aggregate of magnetized nanoparticles (e.g., detection of a large amplitude magnetic field produced by such aggregated magnetized nanoparticles, detection of a shortened decoherence time, T2 time constant, or other properties of magnetic spins of atomic nuclei) allows for the determination that the single instance of the analyte is present (e.g., that a cancer cell is present in a portion of subsurface vasculature). Other properties of a detected magnetic field produced by and/or affected by magnetized nanoparticles could be used in similar or different ways to determine properties of one or more analytes in an environment of interest.

The method 1100 could include additional steps or elements. For example, the method 1100 could include introducing the nanoparticles into the biological environment (e.g., into a portion of subsurface vasculature by injecting, ingesting, transdermally transferring, or otherwise introducing the engineered nanoparticles into a lumen of vasculature of a human). In some examples, the method 1100 could include collecting the magnetized nanoparticles in a portion of subsurface vasculature, e.g., to extract the nanoparticles and/or to increase a magnitude of the magnetic field produced by the magnetized nanoparticles as detected by the magnetometer. The method 1100 could include additional or alternative steps.

### VIII. CONCLUSION

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

While various aspects and embodiments herein are described in connection with detecting magnetic and/or electromagnetic fields produced and/or influenced by magnetized nanoparticles disposed in particular example biological environments (e.g., a portion of subsurface vasculature) to detect and/or determine properties (e.g., a presence, a concentration, a number, a degree of aggregation, a binding state) of the magnetized nanoparticles, other applications and environments are possible. Aspects and embodiments herein could be applied to detect properties of magnetized nanoparticles in in vivo or in vitro human or animal tissues, a fluid in a scientific, medical, or industrial testing process, or some other environment. Properties of magnetized nanoparticles disposed in a natural environment, e.g., a lake, river, stream, marsh, or other natural locale could be detected. Properties of magnetized nanoparticles disposed in a fluid environment of an industrial process or other artificial environment, e.g., a water treatment process, a food preparation process, a pharmaceutical synthesis process, a chemical synthesis process, a brewing and/or distilling process, or other artificial locale could be detected. Other environments and applications of aspects and embodiments described herein are anticipated.

Where example embodiments involve information related to a person or a device of a person, such embodiments may include privacy controls. Such privacy controls may include, at least, anonymization of device identifiers, transparency and user controls, including functionality that would enable users to modify or delete information relating to the user's use of a product.

Further, in situations wherein embodiments discussed herein collect personal information about users, or make use of personal information, the users may be provided with an opportunity to control whether programs or features collect user information (e.g., information about a user's medical history, social network, social actions or activities, profession, a user's preferences, or a user's current location), or to control whether and/or how to receive content from the content server that may be more relevant to the user. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over how information is collected about the user and used by a content server.

What is claimed is:

1. A device comprising:
a magnetometer, wherein the magnetometer is configured to detect magnetic fields at a first location of subsurface vasculature;
a magnetic flux source, wherein the magnetic flux source is configured to magnetize nanoparticles in a second location of subsurface vasculature, wherein the second location is located upstream from the first location relative to a direction of blood flow in the subsurface vasculature; and
a controller operably coupled to the magnetometer, wherein the controller comprises a computing device programmed to perform controller operations comprising:
operating the magnetometer to detect a magnetic field at the first location; and
determining a property of magnetized nanoparticles based on the detected magnetic field, wherein the magnetized nanoparticles include nanoparticles that were magnetized by the magnetic flux source at the second location.

2. The device of claim 1, wherein determining the property of the magnetized nanoparticles based on the detected magnetic field comprises determining a degree of aggregation of the magnetized nanoparticles.

3. The device of claim 1, wherein the controller operations further comprise determining a property of an analyte bound to the magnetized nanoparticles based on the determined property of the magnetized nanoparticles.

4. The device of claim 3, wherein determining the property of the analyte bound to the magnetized nanoparticles comprises determining an amount of the analyte.

5. The device of claim 1, wherein the magnetometer is configured to be positioned on an external body surface proximate the first location of subsurface vasculature, wherein the magnetic flux source is configured to be positioned on an external body surface proximate the second location of subsurface vasculature.

6. The device of claim 1, wherein the magnetometer comprises a spin-exchange relaxation-free atomic magnetometer.

7. The device of claim 1, wherein the magnetometer comprises a multipass scalar atomic magnetometer.

8. The device of claim 1, further comprising an excitation coil, wherein the excitation coil is configured to be positioned proximate to the first location of subsurface vasculature and to produce an oscillating magnetic field in the subsurface vasculature, and wherein operating the magnetometer comprises operating the magnetometer to detect time-varying magnetic fields produced by the magnetized nanoparticles proximate the second location of subsurface vasculature in response to the oscillating magnetic field produced by the excitation coil.

9. The device of claim 1, further comprising a pulse emitter, wherein the pulse emitter is configured to be positioned proximate to the first location of subsurface vasculature and to rotate the magnetic spins of atomic nuclei by producing a time-varying magnetic field in the subsurface vasculature, and wherein operating the magnetometer comprises operating the magnetometer to detect time-varying magnetic fields produced by atomic nuclei in the subsurface vasculature in response to rotation of the spins of the atomic nuclei by the pulse emitter.

10. The device of claim 1, further comprising:
at least one bias coil, wherein the at least one bias coil is configured to produce a bias magnetic field such that the magnetic field detected by the magnetometer is reduced by an amount related to the bias magnetic field, and wherein the controller operations further comprise:
determining a bias field magnitude; and
operating the at least one bias coil to produce the bias magnetic field according to the determined bias field magnitude.

11. The device of claim 1, further comprising:
a permanent magnet, wherein the permanent magnet is configured to produce an offset magnetic field such that the magnetic field detected by the magnetometer is reduced by an amount related to the offset magnetic field, wherein a magnitude and a direction of the offset magnetic field are selected such that the offset magnetic field at least partially cancels a magnetic field produced by the magnetic flux source at the first location.

12. The device of claim 1, wherein the nanoparticles have magnetic relaxation times within a specified range of relaxation times, wherein the specified range of relaxation times is between approximately 1 second and approximately 2 seconds, wherein the first and second locations are separated by a specified distance such that nanoparticles magnetized by the magnetic flux source during a first period of time while in the second location are still substantially magnetized during a second period of time while in the first location.

13. The device of claim 1, wherein the nanoparticles comprise first nanoparticles having magnetic relaxation times within a first specified range of relaxation times and second nanoparticles having magnetic relaxation times within a second specified range of relaxation times, and further comprising:
a further magnetometer, wherein the further magnetometer is configured to be positioned proximate to a third location of subsurface vasculature and configured to detect magnetic fields at the third location, and wherein the third location is located downstream from the first location relative to a direction of blood flow in the subsurface vasculature, and wherein the controller operations further comprise:
operating the further magnetometer to detect a further magnetic field at the third location, wherein determining a property of magnetized nanoparticles based on the detected magnetic field comprises determining a property of magnetized nanoparticles based on the detected further magnetic field.

14. A method comprising:
magnetizing, using a magnetic flux source, nanoparticles in a first location of subsurface vasculature;
detecting, using a magnetometer, a magnetic field at a second location of the subsurface vasculature, wherein the second location is located downstream from the first location relative to a direction of blood flow in the subsurface vasculature; and
determining a property of magnetized nanoparticles based on the detected magnetic field, wherein the magnetized nanoparticles include nanoparticles that were magnetized by the magnetic flux source at the first location.

15. The method of claim 14, wherein determining a property of magnetized nanoparticles based on the detected magnetic field comprises determining a degree of aggregation of the magnetized nanoparticles.

16. The method of claim 14, further comprising:
determining a property of an analyte bound to the magnetized nanoparticles based on the determined property of the magnetized nanoparticles.

17. The method of claim 16, wherein determining a property of an analyte bound to the magnetized nanoparticles comprises determining an amount of the analyte.

18. The method of claim 14, further comprising:
producing an oscillating magnetic field in the second location of subsurface vasculature, wherein detecting a magnetic field at the second location of subsurface vasculature comprises detecting a time-varying magnetic field produced by magnetized nanoparticles proximate the second location of subsurface vasculature in response to the produced oscillating magnetic field.

19. The method of claim 18, wherein detecting a time-varying magnetic field produced by magnetized nanoparticles in response to exposure to the produced oscillating magnetic field comprises detecting a time-varying magnetic field at a frequency that is a multiple of the frequency of the produced oscillating magnetic field.

20. The method of claim 14, further comprising:
rotating the magnetic spins of atomic nuclei by producing a time-varying magnetic field in the second location of subsurface vasculature, wherein detecting a magnetic field in the second location of subsurface vasculature comprises operating the magnetometer to detect time-varying magnetic fields produced by atomic nuclei in the second location of subsurface vasculature in response to rotation of the spins of the atomic nuclei.

21. The method of claim 14, wherein the nanoparticles comprise first nanoparticles having magnetic relaxation times within a first specified range of relaxation times and second nanoparticles having magnetic relaxation times within a second specified range of relaxation times, and further comprising:
detecting, using a further magnetometer, a further magnetic field produced in a third location of subsurface vasculature, wherein the third location is located downstream from the second location relative to a direction of blood flow in the subsurface vasculature, and wherein determining a property of magnetized nanoparticles based on the detected magnetic field comprises determining a property of magnetized nanoparticles based on the detected further magnetic field.
